(19) United States
(12) Patent Application Publication
(10) Pub. No.: US 2013/0224117 A1
(43) Pub. Date: Aug. 29, 2013

(54) LATENT VARIABLE APPROACH TO THE IDENTIFICATION AND/OR DIAGNOSIS OF COGNITIVE DISORDERS AND/OR BEHAVIORS AND THEIR ENDOPHENOTYPES

(71) Applicant: The Board of Regents of the University of Texas System, (US)

(72) Inventors: Donald R. Royall, San Antonio, TX (US); Raymond F. Palmer, San Antonio, TX (US)

(73) Assignee: The Board of Regents of the University of Texas System, Austin, TX (US)

(21) Appl. No.: 13/776,661

(22) Filed: Feb. 25, 2013

Related U.S. Application Data
(60) Provisional application No. 61/603,226, filed on Feb. 24, 2012.

(51) Int. Cl. G06F 19/00; G01N 33/50

(52) U.S. Cl. CPC G06F 19/3437; G01N 33/5088
USPC 424/9.2; 702/19

(57) ABSTRACT

Certain embodiments are directed to methods of distinguish “target-relevant” variance in observed clinical and physiological measures from the variance in observed data that is unrelated to any target process.

This application claims priority to U.S. Provisional Application Ser. No. 61/603,226 filed Feb. 24, 2012, which is incorporated herein by reference in its entirety.

### BACKGROUND

The current “State of the Art” for dementia case-finding is a consensus clinical diagnosis made by experienced clinicians with full access to comprehensive psychometric data, and employing standardized clinical diagnostic criteria (McKhann et al. (1984) Neurology 34, 939-944; Román et al. (1993) Neurology. 43(2):250-60; McKeith et al. (1999) Neurology 53(5):902-05; Winblad et al. (2004) J Intern Med 256:240-246; Carins et al. (2007) Acta Neuropathol. 114(1):5-22. Latent variables can be used in a structural equation modeling (SEM) framework. Latent approaches to the analysis of cognitive test performance (i.e., factor analyses) and to a lesser extent, latent growth curve (LGC) models, are known. A specific latent variable, General Intelligence or “g” has been described since the early 20^{th} century (Spearman (1904) Am J Psychol 15:201-293), although it's relevance to regional brain pathology and dementia has only recently been explored (Duncan et al. (1997) Cogn Neuropsychol 14, 713-741; Duncan and Owen (2000) Trends Neurosci. 23, 475-483; Duncan et al. (2000) Science 289, 457-460; Choi et al. (2008) J Neuroscience 28, 10323-10329; Bouchard (2009) Ann Hum Biol 36, 527-544; Gläscher et al. (2010) PNAS 107, 4705-4709). Latent factor models of neuropsychological test scores have been developed in the aging and Alzhemier's disease (AD) literature (Lowenstein et al., 2001; Chapman et al., 2010; Dowling et al., 2010) but these uniformly combine cognitive measures alone, and rarely attempt to predict clinical consensus diagnoses.

### SUMMARY

Currently case-finding is a consensus clinical diagnosis made by experienced clinicians with full access to comprehensive psychometric data, and employing standardized clinical diagnostic criteria. Such assessments are unwieldy, expensive, burdensome and necessarily limited to tertiary research centers and small sample sizes with limited generalizability. Such a method is unsuitable for studies in rural areas, to large samples, or in minority populations. The methods described herein overcome these limitations. Moreover, the approach results in a continuously varying, measurement error-free dementia endophenotype. This is much more statistically robust than diagnostic categories, which are both categorical, which leads to loss of information, and prone to measurement error. Using the methods described herein, studies can be conducted with improved power, smaller sample sizes, and in minority or difficult to assess target populations.

In certain aspects endophenotype refers to a type of biomarker used in clinical medicine whose purpose is to divide symptoms into a phenotype with a genetic connection. Typically, an endophenotype is (a) associated with illness in the population, heritable, primarily state-independent (manifests in an individual whether or not illness is active), and co-segregates with a state within families.

Certain embodiments are directed to methods that can explicitly distinguish “target-relevant” variance in observed clinical and physiological measures from the variance in observed data that is unrelated to any selected target process. The approach has been validated in the context of dementia assessment but is applicable to other clinical, cognitive, behavioral, and/or functional assessments. In the case of dementia, the method results in a latent variable or score, “d” (dementia-relevant variance in cognitive task performance), that represents only a small fraction of the total variance in observed cognitive task performance, yet is associated with clinicians' assessments of dementia status and severity.

In certain aspects, the covariance between a battery of clinical measures and a target variable related to the diagnosis or outcome of interest are used in a structural equation model to define a hybrid latent variable that represents the targeted outcome. The hybrid latent variable can be used as an endophenotype or to predict conditions or clinical states that are not readily discernable by using variance measures of a first type of assessment (e.g., the clinical battery) alone or a second type assessment (e.g., the target variable) alone. The new hybrid latent variable that is based on the covariance between the variance between the first and second assessments (e.g., a cognitive-functional latent variable) can be included in the structural equation model, scaled, and compared to known outcome(s) to classify an unknown into 1, 2, 3, 4, 5, 6, 7 or more different classifications. The outcome classifications can be defined by available data that has been assessed or classified using one or more other, more restrictive, expensive or impractical methodologies (e.g., Clinical Dementia Rating Scale scores, expert consensus diagnoses, neuroimaging, etc.). In certain aspects, the targeted outcome is a cognitive one and in particular aspects the outcome is dementia, suicidal tendencies, decision-making capacity and the like.

Aspects of the invention can be performed using a remote communication such as email, telephone, web-base questionnaire, and the like. In certain aspects, a technician or other personnel may collect the information needed to executive the program without either experience in, or knowledge of, the interpretation of the measures being used to make these categorizations. Thus, this method frees clinical diagnoses from the need for expert opinion.

Certain embodiments are directed to a method of assessing the dementia status of a subject comprising determining a cognitive-functional correlate score (“d”) indicative of covariance between cognitive performance assessment and functional performance assessments of a subject. In certain aspects, the subject is suspected of having Alzheimer's disease. In certain aspects the cognitive-functional correlate score is assessed by comparison with a scale that is determined using data that has been classified into at least 2 outcomes, e.g., normal and dementia. In certain aspects the scale can be sub-classified in to 1, 2, 3, 4, 5, 6, 7, or more outcomes that can be determined subjectively or objectively using cognitive and functional data.

Certain embodiments are directed to a method of identifying one or more biomarkers of a targeted condition comprising: (a) measuring levels of a plurality of biomarkers in a biological sample from a group of subjects; (b) determining a cognitive-functional correlate score related to the covariance between their cognitive and their functional assessment, wherein the score identifies subjects with a higher likelihood of having a cognitive condition; and (c) identifying one or more biomarkers having levels that correlate with the resulting hybrid latent construct, e.g., dementia, or simply “d”.

Further embodiments are directed to a method for evaluating the effectiveness of a therapeutic comprising: (a) determining a first cognitive-functional correlate score indicative of covariance between cognitive performance assessment and functional performance assessments of a subject; (b) administering a therapeutic to a subject; (c) determining a second cognitive-functional correlate score indicative of covariance between cognitive performance assessment and functional performance assessments of a subject; and (d) comparing the first and second cognitive-functional correlate scores, wherein a relative change in the first and second cognitive-functional correlate score is indicative of the effectiveness of the therapeutic.

The approach described herein also results in a measurement “error-free” continuously varying endophenotype of the targeted condition (e.g., dementia) that can be used to make accurate clinical diagnoses from limited psychometric batteries, and can be used as an outcome in studies of potential biomarkers. The methods described are free of cultural, linguistic, or educational bias, and can be employed with very limited datasets, using either existing measures, or easily collected ones (i.e., telephone measures or brief screening tests). Moreover, the approach is modular, and can be easily adapted to multiple target conditions, potentially including, but not limited to, aging, depression, schizophrenia, or other difficult to assess conditions.

This method can be used to replicate the expert consensus diagnoses of experienced clinicians from telephone assessments, small psychometric batteries, or routine blood tests, or to identify specific biomarkers of target conditions.

The newly developed variable d correlates strongly (partial r=0.80-0.96) with current consensus dementia severity measures (i.e., the Clinical Dementia Rating Scale (CDR) (Hughes et al., 1982), and is highly accurate in predicting the consensus clinical diagnoses of experienced clinicians [Receiver Operating Curve (ROC) Area Under the Curve (AUC)=0.96-0.99 for the discrimination between Alzheimer's Disease (AD) and controls]. As a latent variable, d's existence may not be obvious to clinicians because it cannot be directly measured. Moreover, the latent construct represented by d comprises only a fraction of the variance in each measure's raw score. However, the fraction of variance in raw cognitive performance that is related to d is strongly related to clinicians' opinions of dementia severity. The individual measures that comprise d each contain unrelated variance and measurement error, which can weaken their unadjusted associations with the CDR. Because the current “State of the Art” is to build such multivariate regression models of dementia status, d's existence has been missed.

The latent variable d's existence may also have escaped detection because it is associated with the Default Mode Network (DMN) (FIG. 11). The DMN is a network of brain regions that are active when the individual is not focused on the outside world and the brain is at wakeful rest. DMN is characterized by coherent neuronal oscillations at a rate lower than 0.1 Hz (one every ten seconds). During psychometric evaluation, the DMN is deactivated and another network, the task-positive network (TPN) is activated. Therefore, the DMN's function(s) are poorly assessed by raw psychometric performance. Thus, d accounts for only a small fraction of the variance in observed psychometric measures, and it is not discernable by their inspection. This aspect is specific to the d endophenotype.

Because of this interesting property, the DMN is “anti-correlated” with task-specific cortical activations (Uddin et al., 2009). Thus, cognitive testing reduces DMN activity. This suggests a fundamental limitation on the ability of cognitive measures to accurately diagnose dementia on their own. The key network cannot be easily interrogated by cognitive tasks. This also explains why d accounts for such a small proportion of overall cognitive variance. The latent variable d's exceptional ability to replicate clinicians' dementia diagnoses may stem from its ability to detect pathology in this key network. The DMN's hubs are specifically targeted by β-amyloid deposition (Buckner et al. (2009) Neuron 63(2):178-88; Buckner et al. (2009) J Neurosci 29(6):1860-73). Tauopathy in the same hubs is strongly associated with clinical dementia (Royall et al. (2002) Exp Aging Res 28(2):143-62).

Although latent variables have been used to analyze cognitive batteries, this has been limited to “g” and secondary factor studies containing only one type of indicator variables (i.e., cognitive measures or functional status measures only). Similarly, although biomarkers have been sought in AD cohorts, only clinical diagnoses have been used as outcomes (i.e., O'Bryant et al. (2008) Arch Neurol 65, 1091-95), never latent variable proxies for clinical diagnoses.

The methods described are not limited to cognitive assessments and/or dementia diagnoses. The methods can be applied to other clinical conditions, and to non-cognitive batteries. For example, if applied to commonly available serum analyte panels, can identify diagnostic blood tests for AD, depression, alcoholism, schizophrenia, or any desired target condition, including functional capacities, such as driving, finance, and medication management, or clinical risks states, such as for suicide or falls, etc.

Certain aspects are directed to methods of evaluating a data set having a first and second type of assessment by including in a structural equation model a hybrid variable that is related to the covariance between the variance of the first and second type of assessment, wherein the hybrid variable is used to determine a score that is compared to a known scale to classify an outcome. In certain aspects the assessment is of dementia status of a subject comprising a hybrid variable defined as a cognitive-functional correlate score (“d score”) that is indicative of covariance between a first cognitive and a second functional status performance assessments of a subject. The known scale can be an optimal d score for diagnosis of Alzheimer's disease, mild cognitive impairment (MCI), and normal cognition calculated from a validation cohort. A “validation cohort” refers to a group of people sharing similar characteristics. Characteristics may include, for example, physical characteristics, presence or absence of a condition or conditions, age, geographic location and the like. The cohort may be defined by the person conducting the research study and a research study may include one or more cohorts. For example, a researcher may be researching the effect of a particular drug. In certain aspects the group of people are used to validate a particular model, such as the structural equation models described herein. This group of people are a validation cohort. Typically, a validation cohort will comprise a range of outcomes that defines the spectrum of conditions to be assessed. In certain aspects the validation cohort has been characterized by known system or diagnostic methodology, thus the outcome of the individuals in the cohort is known. With the validation cohort established an uncharacterized individual can be assessed and compared to the spectrum or scale produced by analysis of the validation cohort.

Other aspects are directed to methods for evaluating the effectiveness of a therapeutic comprising (a) determining a first cognitive-functional correlate score indicative of covariance between cognitive performance assessment and functional performance assessments of a subject; (b) administering a therapeutic to a subject; (c) determining a second cognitive-functional correlate score indicative of covariance between cognitive performance assessment and functional performance assessments of a subject; and (d) comparing the first and second cognitive-functional correlate scores, wherein a relative change in the first and second cognitive-functional correlate score is indicative of the effectiveness of the therapeutic.

In certain aspects, methods comprising constructing, by a computing device, a score based on a hybrid latent variable generated by a structural equation model that is related to the covariance of two or more assessment measure variances; and classifying one or more outcome based on comparing the constructed score to a known scale constructed from scores of a validation cohort. The hybrid latent variable score can be a cognitive-functional latent variable score (d score).

In certain aspects, methods for assessing a condition in an individual comprise: (a) selecting (i) a battery of behavioral measures of a subject, and (ii) one or more measures of a target condition or disease; (b) constructing (i) a first latent factor related to variance of the behavioral measures, (ii) a second latent factor related to variance of the target measures, and (iii) a third hybrid factor related to covariance of the behavioral measures and the target measures by using structural equation modeling (SEM); (c) determining the hybrid factor loadings on a validation cohort and using the loading to export a score for each individual in a validation cohort; (d) selecting score thresholds based on the validation cohort; (e) applying the score threshold to a score obtained from the individual being assessed, wherein the score for the individual is obtained by administering the same set of measures used to construct the hybrid factor in the validation cohort where the individual's score is compared to the score thresholds of the validation cohort. In certain aspects the score thresholds define those subjects with dementia, mild cognitive impairment, or normal cognition. The behavioral measure can comprise a battery of verbal measures, a battery of non-proprietary measures, a battery of bedside measures or any other measures that can be related to a particular target. The target condition or disease can be a diagnosis, propensity to develop a condition, mood state, behavior, or biomarker related to a condition or disease. In certain aspects the optimal score thresholds are selected by Receiver Operating Curve (ROC) analysis of determinations of the same population used to construct the hybrid latent factor. In certain aspects the operations for applying the method are at least in part executed on a phone, tablet, computer, or internet-based server.

As used herein, the term “biomarker” or “biochemical marker” refers to a protein, nucleic acid, or metabolite that is to be measured, detected, analyzed biochemically and/or monitored, for example, a small molecule, RNA, antigen, or antibody.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions, kits, and computer software of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustration of a structural equation model (SEM) of two latent factors: “g” and “f”. Observed variables are represented by rectangles, while latent constructs are represented by circles. Arrows reflect regression weights, or factor loadings in the case of a latent variable's indicators. Bidirectional arrows represent correlations. ADL=Basic Activities of Daily Living; CDR=Clinical Dementia Rating scale sum of boxes; COWA=Controlled Oral Word Association Test; DST=Digit Span Test; IADL=Instrumental Activities of Daily Living; WMS LM II=Weschler Memory Scale: Delayed Logical Memory; WMS VR II=Weschler Memory Scale: Delayed Visual Reproduction. *All observed variables are adjusted for age, gender and education. Residuals and their inter-correlations not shown.

FIG. 2. Illustration of a structural equation model (SEM) of two latent factors: “g” and “f” including the third latent variable “d”. Observed variables are represented by rectangles, while latent constructs are represented by circles. Arrows reflect regression weights, or factor loadings in the case of a latent variable's indicators. Bidirectional arrows represent correlations. ADL=Basic Activities of Daily Living; CDR=Clinical Dementia Rating scale sum of boxes; COWA=Controlled Oral Word Association Test; DST=Digit Span Test; IADL=Instrumental Activities of Daily Living; WMS LM II=Weschler Memory Scale: Delayed Logical Memory; WMS VR II=Weschler Memory Scale: Delayed Visual Reproduction. *All observed variables are adjusted for age, gender and education. Residuals and their inter-correlations not shown. CDR SOB (Model 2a), MMSE (Model 2b), and GDS (Model 2c) modeled separately (Table 4), and combined in this figure.

FIG. 3. Histogram of d scores respectively. d scores are bimodally distributed, as is the TARCC sample itself, which was composed of “dementia cases” and “controls.”

FIG. 4. Histogram of g′ scores. g′ scores, a sizable fraction of the cognitive battery's total variance, are normally distributed because g′, unlike d, is orthogonal to dementia status.

FIG. 5. Illustration of regional grey matter atrophy is associated with d, adjusted for g′, f, age, gender and education (BAP).

FIG. 6. Contrasts digit span (DST), verbal fluency (COWA), Boston Naming (Boston), visual recall (VRII), and paragraph recall (LMII). This battery sorts itself out into measures that approach d's accuracy in detecting dementia (e.g., LMII and VRII) and those that do not (e.g., Boston, COWA, DSS). In fact, each measure's rank ordered AUC recapitulates its rank ordered loading on d.

FIG. 7. Illustrates a model of d derived from only three cognitive measures (Immediate and Delayed Paragraph Recall from the Weschler Memory Scale) and category fluency (Animals).

FIG. 8. Illustrates that the use of the PSMS disadvantages the model slightly, due to lack of frank dementia cases, and thus of cases with impairment in BADL's

FIG. 9. Illustrates the comparison to the larger TARCC cohort which does not contain the AQ.

FIG. 10. Demonstrates that the measures that define d are not so strongly associated with dementia severity as d itself.

FIG. 11. Illustrates the mapping of d to DMN hubs.

FIG. 12. Illustrates a block diagram of a computer system configured to implement various systems and methods described herein according to some embodiments.

FIG. 13. Model 1*-d Correlates Strongly with CDR-SB. *All observed variables adjusted for age, gender and education (not shown). Animals, Category Fluency: Animals; Boston, Boston Naming Test (15 item); CDR-SB, Clinical Dementia Rating Scale Sum of Boxes; LMIIA, Wechsler Memory Scale—Revised Logical Memory Story A Delayed; MCI-ADL, Alzheimer's Disease Cooperative Study Activities of Daily Living Scale for Mild Cognitive Impairment; SRTFR, Selective Reminding Task Free Recall Total; StrI, Stroop Color Task Color-Word Interference Task.

FIG. 14. Model 2*; DEPCOG Correlates Strongly with CDR-SB. *All observed variables adjusted for age, gender and education (not shown). Animals, Category Fluency: Animals; Boston, Boston Naming Test (15 item); CDR-SB, Clinical Dementia Rating Scale Sum of Boxes; GDSs, Geriatric Depression Scale Subject rated; LMIIA, Wechsler Memory Scale—Revised Logical Memory Story A Delayed; MCI-ADL, Alzheimer's Disease Cooperative Study Activities of Daily Living Scale for Mild Cognitive Impairment; SRTFR, Selective Reminding Task Free Recall Total; StrI, Stroop Color Task Color-Word Interference Task.

FIG. 15. Model 3*; The Latent Variables d and DEPCOG Contribute Independently to Diagnosis. *All observed variables adjusted for age, gender and education (not shown). Animals, Category Fluency: Animals; Boston, Boston Naming Test (15 item); CDR-SB, Clinical Dementia Rating Scale Sum of Boxes; GDSc, Geriatric Depression Scale Caregiver rated; LMIIA, Wechsler Memory Scale—Revised Logical Memory Story A Delayed; MCI-ADL, Alzheimer's Disease Cooperative Study Activities of Daily Living Scale for Mild Cognitive Impairment; SRTFR, Selective Reminding Task Free Recall Total; StrI=Stroop Color Task Color-Word Interference Task.

FIG. 16. Model 4*; Symptom Content Mediates the GDS' Effect. *All observed variables adjusted for age, gender and education (not shown). Animals, Category Fluency: Animals; Boston, Boston Naming Test (15 item); CDR-SB, Clinical Dementia Rating Scale Sum of Boxes; GDSc, Geriatric Depression Scale Caregiver rated; LMIIA, Wechsler Memory Scale—Revised Logical Memory Story A Delayed; MCI-ADL, Alzheimer's Disease Cooperative Study Activities of Daily Living Scale for Mild Cognitive Impairment; SRTFR, Selective Reminding Task Free Recall Total; StrI=Stroop Color Task Color-Word Interference Task.

FIG. 17. Scatterplot of DEPCOG factor against d factor.

FIG. 18. Regional cortical atrophy associated specifically with DEPCOG*. *Adjusted for age and gender (and implicitly for education and g′). Note overlap with elements of the Default Mode Network. Regional cortical volume associated with δ (left column), d (middle left column), and DEPCOG (middle right column). Each analysis is adjusted for age, gender, and education (and implicitly for g′). The bar represents the voxel-wise T statistic, only significant voxels are presented (FWE>0.05, k>50). The overlap of the maps can be seen in right column

FIG. 19. A posterior cingulate (PCC) seed Replicates d and DEPCOG*. *Adjusted for age and gender (and implicitly for education and g′). Regional cortical volume associated with volume in the posterior cingulate (PCC seed, left column), d (middle left column), and DEPCOG (middle right column). The bar represents the voxel-wise T statistic, only significant voxels are presented (FWE>0.05, k>50). The overlap of the maps can be seen in right column.

FIG. 20. dMA* in MA TARCC Subjects. CDR-SB=Clinical Dementia Rating Scale Sum of Boxes; CLOX1=Unprompted clock drawing from CLOX: An Executive Clock-Drawing Task; CLOX2=copied clock drawing; HSWK=housework IADL item; IADL=InstrumentalActivities of Daily Living; MA=Mexican-American; MMSE=Mini-Mental Status Exam; MONY=financial management IADL item; TARCC=Texas Alzheimer's Research and Care Consortium. *All indicator variables are additionally adjusted for age, gender and education (not shown for clarity).

FIG. 21. ROC Analysis of AD v MCI+Controls in MA Subjects.

FIG. 22. Illustration of various modules that can be used to implement embodiments of the invention.

FIG. 23. Illustration of one embodiment of implementing aspects of the invention.

### DESCRIPTION

Cognitive impairment is widely held to be the hallmark of dementia. However, three conditions are necessary to that diagnosis (Royall et al. (2007) J Neuropsychiatry Clin Neurosci 19, 249-265): (1) there must be acquired cognitive impairment(s), (2) there must the functional disability, and (3) the disability must be related to the cognitive impairment(s) that are observed. This implies that the essential feature(s) of dementing processes can be resolved to the cognitive correlates of functional status.

Psychometric and informant-based clinical measures are notoriously prone to measurement error, particularly in minority populations with limited educational attainment and culture-linguistic barriers to their assessment. Latent variable “measurement models” (Cook et al. (2001) Soc Sci Med 53(10):1275-85) offer the potential for “error free” measures of key constructs. A latent variable model is described herein that provides both a measure of dementia severity and a continuously varying “error free” dementia-specific endophenotype. By using both cognition and functional status measures as indicators, the inventors have achieved an unprecedented ability to model dementia status from easily acquired datasets.

Target-related outcome variables can be mixed with a battery of predictors to “distill” or “refine” their shared variance into a latent variable of interest. The factor scores of the resulting latent construct can be output to create an error free continuously varying endophenotype, which can then be used as an outcome variable or predictor in its own right.

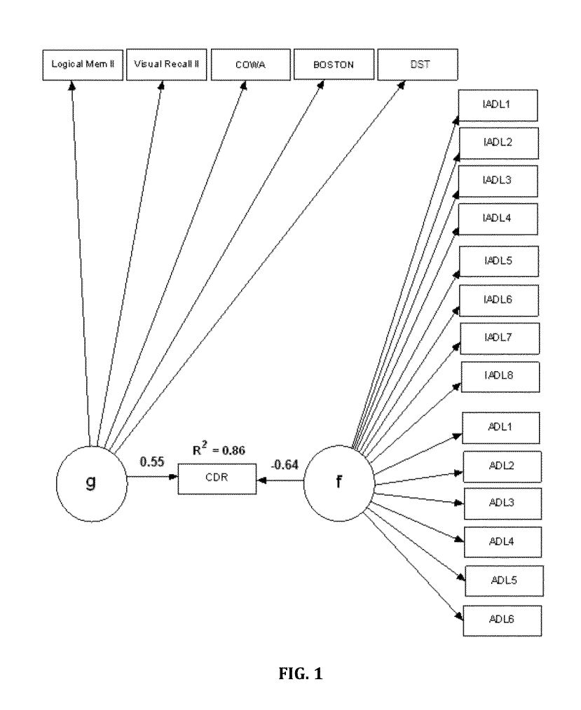

FIG. 1 presents a structural equation model (SEM) of two latent factors: “g” and “f”. In SEM, observed variables are represented by rectangles, while latent constructs are represented by circles. Arrows reflect regression weights, or factor loadings in the case of a latent variable's indicators. Bidirectional arrows represent correlations. The latent variable g represents “Spearman's g”, i.e., a latent variable representing the shared variance across the observed cognitive performance variables (Spearman (1904) Am J Psychol 15:201-293). In data from the Texas Alzheimer's Research and Care Consortium (TARCC), g explains 68.8% of the variance in observed psychometric performance. F represents a latent functional status factor derived from eight observed instrumental activities of daily living (IADL) items and six observed basic ADL (BADL) items. The latent variable f explains 50.67% of the variance in observed variance in care-giver rated IADL/BADL.

The observed cognitive measures all loaded significantly on g (range: r=−0.65-−0.79; all p<0.001). LM II loaded most strongly (r=−0.79). Digit Span loaded least strongly (r=−0.65). The observed IADL/BADL items all loaded significantly on f (range: r=−0.37-−0.84; all p<0.001) (Table 2). Shopping and responsibility for medication adherence loaded most strongly (both r=−0.84). Toileting loaded least strongly (r=−0.37).

In a multivariate regression (FIG. 1), g and f were each strong, significant, and independent predictors of CDR SOB. Together, g and f explained 86% of the variance in CDR scores. Nonetheless, the model did not fit adequately well. Significant inter-correlations amongst the residuals (not shown in FIG. 1) support the existence of an additional latent variable.

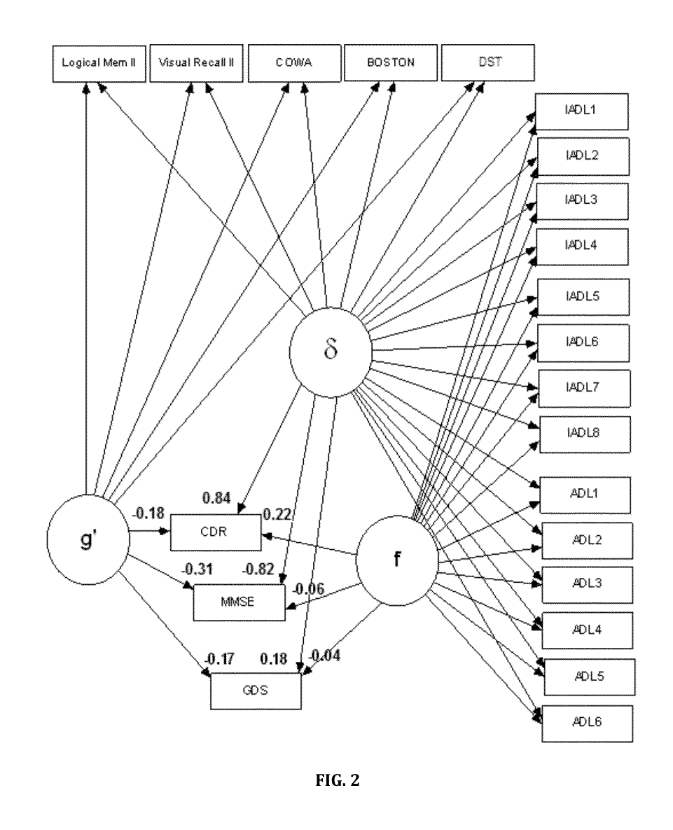

A third latent variable, a hybrid cognitive/functional status latent construct, is introduced “d” (FIG. 2). The latent construct d represents the variance shared between cognitive and IADL/BADL measures [i.e., any and all dementing process(es) afflicting the sample]. The creation of d attenuated the association between g and several measures of cognitive performance (range r=0.32-0.48; all p<0.001). The inventors relabeled g as “g′” to acknowledge this effect. Together, g′ and d accounted for 59.6% of the variance in our cognitive battery. The latent construct d accounted for 37.2% independently of g′. The remainder was attributable to residual “measurement error”.

The latent construct f was also affected by the creation of d. The latent construct f retained relatively strong associations with the BADL items (range r=0.35-0.62, all p<0.001) but lost its formerly strong associations with IADL items (range r=0.10-0.28), one of which (cooking) no longer loaded significantly on f (r=0.10, p=0.068). This shows that IADL items are more relevant to dementing illness (through d) than are BADL items.

The latent construct d was significantly and inversely associated with each cognitive performance measure (range: r=−0.55-−0.67; all p<0.001). It was most strongly associated with WMS VRII (r=−0.67), and least strongly associated with DST (r=−0.55).

The latent construct d was also strongly and positively associated with each IADL item (range: r=0.51-0.87). The latent construct d was most strongly associated with shopping (r=0.87) and least strongly associated with laundry (r=0.51). Each BADL item loaded significantly (and positively) on d, but the strength of these associations was relatively weak (range: r=0.25-0.56). The latent construct d was most strongly associated with ADL4 (grooming) (r=0.56) and least strongly associated with ADL 1 (toileting) (r=0.25). Thus, in contrast to f in FIG. 1, d appears to be relatively specifically related to variance in IADL and not BADL items.

As a test of d's construct validity, the inventors regressed the base model of g′, d, and f onto CDR SOB (FIG. 2). Together, g′, f, and d explained 90% of the variance in CDR SOB. However, this was almost entirely mediated by d (r=0.84; p<0.001). In contrast to FIG. 1, g's association was severely attenuated, but remained significant (r=−0.18; p=<0.001). The latent construct f's former association with dementia severity was also attenuated (partial r=0.22; p<0.001).

Discriminant validity is provided by multivariate regression models of Mini-Mental State Exam (MMSE) (Folstein et al. (1975) J Psychiatr Res 12, 189-198) and Geriatric Depression Scale (GDS) (Sheikh and Yesavage (1986) Clin Gerontologist 5, 165-173) scores (FIG. 2). The MMSE is a measure of global cognition and should be more strongly associated with a dementing process than the GDS, a measure of depressed mood. As expected, d's association with these measures was weakened relative to that with CDR SOB. g's association with MMSE scores was strengthened relative to that with CDR SOB. g′ and d were weakly associated with GDS scores. The latent construct f did not contribute significantly to either of those outcomes.

The latent variables g′, f, and d were tested as independent predictors of TARCC consensus clinical diagnoses (i.e., “AD” vs. “control”). The latent construct d achieved the most accurate discrimination (AUC=0.942). The latent construct g′ (AUC=0.790) was more accurate in this discrimination than was f (AUC=0.550). When CDR scores were dichotomized about a threshold of 1.0, d again achieved the most accurate discrimination (AUC=0.996).

The latent variables d and g′ can be output as case-wise factor scores. d scores uniquely can be used as a dementia endophenotype. Similarly, homologs of d created from other target indicator variables can be output as endophenotypes of their respective target conditions (e.g., age, depression, gender, schizophrenia, alcoholism, mortality, etc.).

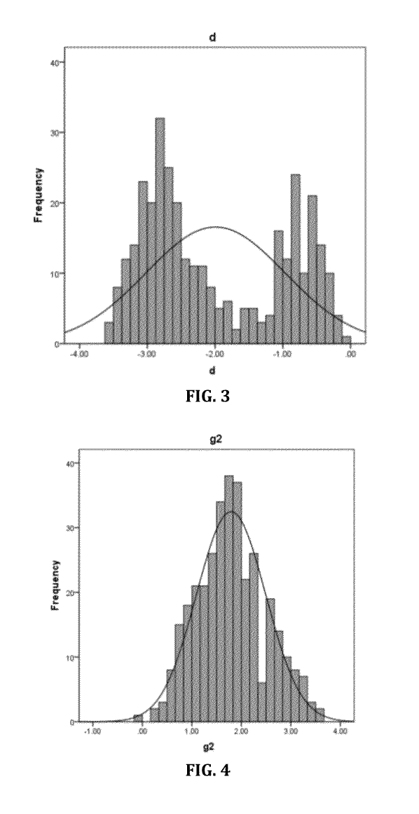

FIGS. 3 and 4 present histograms of d and g scores respectively. d scores are bimodally distributed, as is the TARCC sample itself, which was composed of “dementia cases” and “controls”. In contrast, g′ scores, a sizable fraction of the cognitive battery's total variance, are normally distributed because g′, unlike d, is orthogonal to dementia status.

Endophenotype Applications:

Once an endophenotype has been created, it can be used as an outcome variable, a predictor, or to make categorical classifications (e.g., diagnoses). In this instance, d scores are used to identify AD-related structural changes associated with d, and therefore with dementia. Having identified those changes, one can use brain imaging to predict d scores, and therefore diagnose dementia from a brain scan.

A Dementia-Endophenotype:

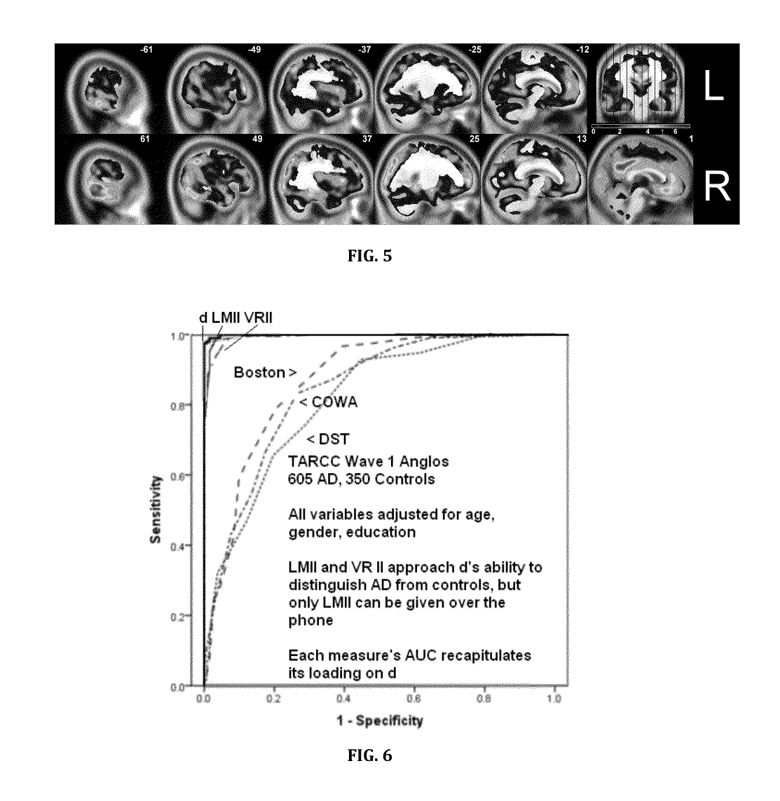

d's factor scores can be exported as a “d score”. This then becomes a continuously varying dementia specific endophenotype. Thus, the interindividual variability in dementia status can be modeled, i.e., as predictors in biomarker studies. FIG. 5 represents regional grey matter density related specifically to d, after adjusting for g′, f, age, gender, and education, among N=23 AD, 47 MCI cases and N=76 controls in the University of Kansas Brain Aging Project (BAP). d's AUC for the discrimination between AD and controls in this sample is 0.987, and AUC=0.955 for the discrimination between MCI and AD. d maps to elements of the Default Mode Network (DMN), which has recently been associated with AD (Buckner et al. (2005) J Neurosci 25(34):7709-17).

Because d scores can be used to effectively rank order each individual in a cohort with respect to their relative position along a dementia-specific continuum, ROC analysis can be used to define optimal empirical d score boundaries for “normal cognition”, “MCI” and “dementia.” Thus, d scores derived from relatively simple batteries could be used to replicate the diagnoses made by experienced clinicians with full access to comprehensive psychometric data. Moreover, this can be applied to any latent d score homolog. Depression, schizophrenia, alcoholism etc. could be accurately diagnosed by the same approach.

d Model Variations:

Because Spearman's g is insensitive to the measures employed in the battery, d can be derived from any desired panel of measures, i.e., measures chosen for their ease of administration, to avoid copyright controls, to reduce respondent burden, or to achieve telephone administration. Moreover, because the latent construct d is an error-free construct, it is not vulnerable to factors such as ethnicity, education, or language of administration, which potentially bias the individual measures used to create it.

Validation of a Potential Telephone-Based d Assessment in Hispanic Cases:

To achieve a telephone application, the inventors first modeled the ability of each cognitive measure in TARCC's psychometric battery to predict clinical consensus dementia status (control vs. AD) relative to d, in ROC analyses. FIG. 6, for example, contrasts digit span (DST), verbal fluency (COWA), Boston Naming (Boston), visual recall (VRII), and paragraph recall (LMII). This battery sorts itself out into measures that approach d's accuracy in detecting dementia (e.g., LMII and VRII) and those that do not (e.g., Boston, COWA, DSS). In fact, each measure's rank ordered AUC recapitulates its rank ordered loading on d.

Because Spearman's g is insensitive to the measures employed in the battery, the inventors can select the assessment to be pursued. Of those that approach d's AUC, paragraph recall (LMII), and category fluency (animals) have the strongest loadings, and can also be administered over the phone.

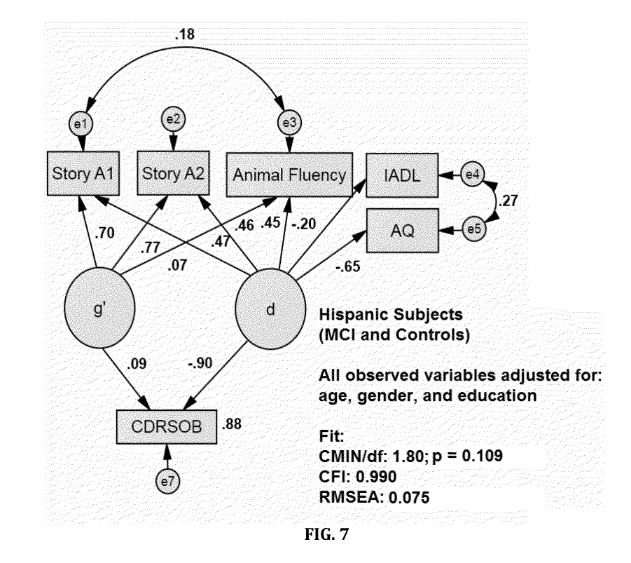

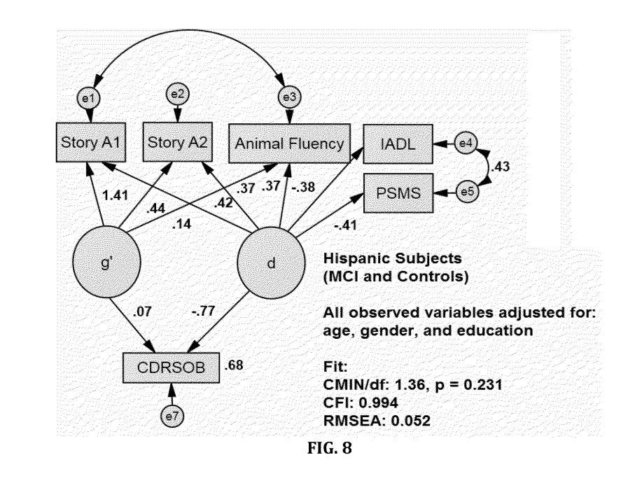

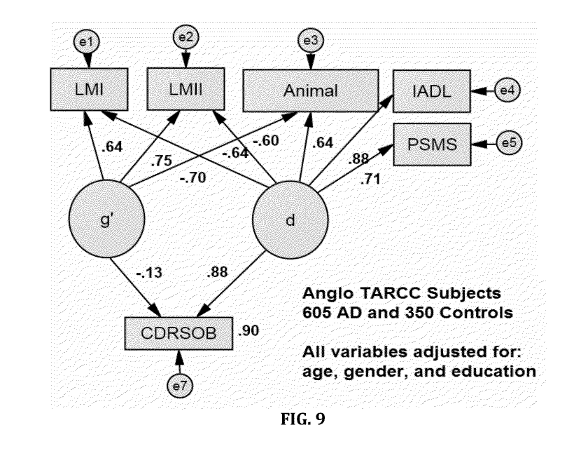

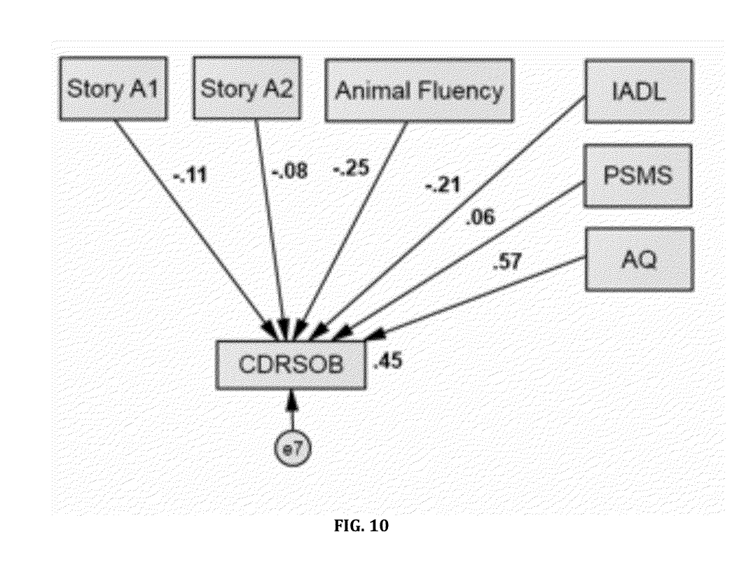

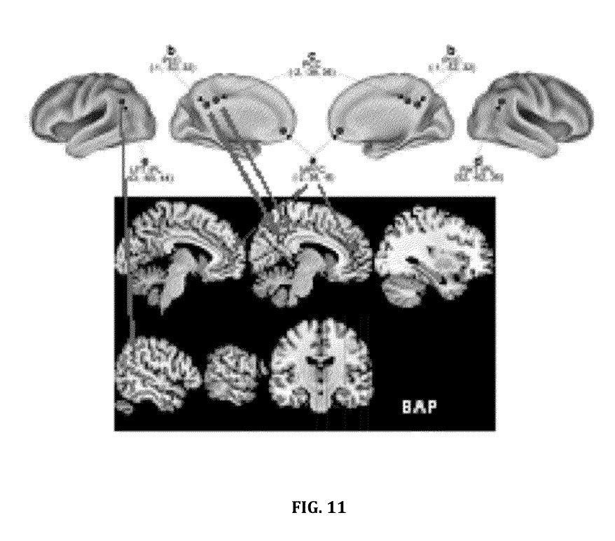

FIG. 7 presents a model of d derived from three cognitive measures (Immediate and Delayed Paragraph Recall from the Weschler Memory Scale) and category fluency (Animals). Both scales load strongly on d in the larger TARCC cohort (N=955 Anglos) and have large AUC's for the discrimination of dementia cases from controls in TARCC (FIG. 6). This battery also includes informant-rated functional status measures (AQ, IADL and PSMS). Out of these measures, the inventors have constructed two latent variables representing g′ and d, and used them to predict CDRSOB in N=80 Hispanic controls vs. 55 non-demented Hispanic cases with MCI. All models are adjusted for age, education, and gender and all achieve excellent fit. In the first model, AQ is used instead of PSMS scores. The use of the PSMS disadvantages the model slightly (FIG. 8), due to the lack of frank dementia cases, and thus of cases with impairment in BADL's. However, this allows for a comparison to the larger TARCC cohort (FIG. 9), which does not contain the AQ. In each case, d disproportionately accounts for the majority of variance in CDRSOB, independently of g′ and the covariates. It is not disadvantaged in Hispanics with relatively poor educational attainment relative to a predominantly Anglo sample. Nor is it disadvantaged by the relatively small sample size in the Hispanic sample, nor by its lack of frankly demented cases.

Thus d, derived solely from a selection of measures that can be obtained over the telephone, is accurately predicting the blinded impressions of experienced clinicians after comprehensive in-person examinations.

### I. DETERMINING A TARGET SCORE USING A HYBRID LATENT VARIABLE

Factor analysis is a statistical method used to describe variability among observed, correlated variables in terms of a potentially lower number of unobserved variables called factors. In other words, it is possible, for example, that variations in three or four observed variables mainly reflect the variations in fewer unobserved variables. Factor analysis searches for such joint variations in response to unobserved latent variables. The observed variables are modeled as linear combinations of the potential factors. The information gained about the interdependencies between observed variables can be used later to reduce the set of variables in a dataset. Computationally this technique is equivalent to low rank approximation of the matrix of observed variables. Factor analysis originated in psychometrics, and is used in behavioral sciences, social sciences, marketing, product management, operations research, and other applied sciences that deal with large quantities of data. Latent variable models, including factor analysis, use regression modeling techniques to test hypotheses. The factor loadings are the correlation coefficients between the variables and factors. Analogous to Pearson's r, the squared factor loading is the percent of variance in that indicator variable explained by the factor. To get the percent of variance in all the variables accounted for by each factor, add the sum of the squared factor loadings for that factor and divide by the number of variables.

In certain embodiments method of determining a score based on a hybrid latent variable can include one or more of the following operations. In certain aspects these operations are executed in part by instructions provided in a tangible medium, such as a programmed computer; a network comprising one or more programmed computers; or a compact disk.

First, select a battery of behavioral measures. There must be at least three. They can be any mix of cognitive and/or behavioral measures, preferably continuously distributed, but not necessarily. The selection of behavioral indicators can be selected in order to achieve a particular application. In certain aspects, a battery of verbal measures would be selected to achieve telephonic administration. In other aspects, a battery of non-proprietary measures might be used to achieve low cost administration. In certain aspects, a battery of bedside measures might be selected to allow data collection by low level psychometricians in the field. A specific battery might be selected to allow post-hoc evaluation of an existing dataset.

Second, select a target. It can be any condition, diagnosis, mood state, behavior or biomarker related to the brain/behavior measures in the battery.

Third, select one or more measures of the target. It can be a battery of measures, or a single measure. Target measure(s) can be selected to achieve the same application(s) as the battery.

Fourth, using Structural Equation Modeling (SEM) methods, construct a latent factor indicated by the measures of the battery. In the case of cognitive measures, this will be an example of Spearman's latent intelligence factor “g”.

Fifth, if the target is being defined by a battery of three or more measures, construct a latent factor indicated by the measures of the battery. In the case of functional status measures, this can be labeled “f”.

Sixth, construct a hybrid factor to be indicated by each measure in the battery and also by the measure(s) of the target measures. In the case of a cognitive performance/functional status hybrid, the resulting latent variable will represent “the cognitive correlates of functional status” and is a proxy for dementia severity (i.e., “d”).

Seventh, the creation of d robs g of some of its variance, altering it's factor loadings. A factor such as g should be re-labeled g′ to acknowledge this change.

Eighth, d's factor loadings (or those of d's ortholog in the case of other targets) can be used to export a “d score” for each individual in the validation cohort. In the case of d, this is a continuously distributed measure of dementia severity. It can be used either as a predictor or an outcome in muItivariate regression or other models (i.e., to determine d's biomarkers or to predict dementia-related clinical outcomes).

Ninth, if d scores (or those of d's ortholog in the case of other targets) are to be used to estimate clinical diagnoses, then an optimal d score threshold must be selected by Receiver Operating Curve (ROC) analysis of expert determinations of that diagnosis in the same population used to construct d.

Tenth, once the optimal threshold has been selected and its accuracy established, the threshold can be applied dichotomously to the d score obtained in any individual unknown case.

Eleventh, to obtain the d score in the unknown case, they are first administered the same set of measures used to construct d in the validation cohort. The scores are entered into a computer program that encodes d's factor loadings. The program is executed on a suitable platform (phone, tablet, computer, or internet-based server). The unknown case is assigned a d score. The d score is compared to the validated reference threshold.

Certain embodiments include the analysis of various cognitive assessment tests and functional assessment tests. The following provide examples of some of the tests that may be provided in isolation or included in a cognitive testing battery. One skilled in such assessments will recognize that other known and novel tests may be applied or used with the methods described herein. Additionally, the tests may be grouped into specific classifications and groups. The collection and arrangement of tests in a battery may be in accordance with a particular cognitive limitation or other criterion. One of skill in such assessments will recognize that the specific tests may be altered and substituted without affecting the novelty of the methods described herein, as may the groupings and ordering of the tests within a test battery.

### II. BIOMARKERS

Biomarkers can be used to both define a disease state as well as to provide a means to predict physiological and clinical manifestations of a disease. Three commonly discussed ways in which biomarkers could be used clinically are: (1) to characterize a disease state, i.e. establish a diagnosis, (2) to demonstrate the progression of a disease, and (3) to predict the progression of a disease, i.e. establish a prognosis. Establishing putative biomarkers for such uses typically requires a statistical analysis of relative changes in biomarker expression either cross-sectionally and/or over time (longitudinally). For example, in a state or diagnostic biomarker analysis, levels of one or more biomarkers are measured cross-sectionally, e.g. in patients with disease and in normal control subjects, at one point in time and then related to the clinical status of the groups. Statistically significant differences in biomarker expression can be linked to presence or absence of disease, and would indicate that the biomarkers could subsequently be used to diagnose patients as either having disease or not having disease. In a progression analysis, levels of one or more biomarkers and clinical status are both measured longitudinally. Statistically significant changes over time in both biomarker expression and clinical status would indicate that the biomarkers under study could be used to monitor the progression of the disease. In a prognostic analysis, levels of one or more biomarkers are measured at one point in time and related to the change in clinical status from that point in time to another subsequent point in time. A statistical relationship between biomarker expression and subsequent change in clinical status would indicate that the biomarkers under study could be used to predict disease progression.

Results from prognostic analyses can also be used for disease staging and for monitoring the effects of drugs. The prediction of variable rates of decline for various groups of patients allows them to be identified as subgroups that are differentiated according to disease severity (i.e. less versus more) or stage (i.e. early versus late). Also, patients treated with a putative disease-modifying therapy may demonstrate an observed rate of cognitive decline that does not match the rate of decline predicted by the prognostic analysis. This could be considered evidence of drug or treatment efficacy.

Various multi-analyte type analyses have been described, for example, WO 2004/104597, “Method for Prediction, Diagnosis, and Differential Diagnosis of AD” describes methods of predicting disease status via an x/y ratio of Aβ peptides; WO 2005/047484, “Biomarkers for Alzheimer's Disease” describes a series of markers that can be used for the assessment of disease state; WO 2005/052592, “Methods and Compositions for Diagnosis, Stratification, and Monitoring of Alzheimer's Disease and Other Neurological Disorders in Body Fluids” teaches methods and markers gleaned from plasma for the monitoring of Alzheimer's disease; and WO 2006/009887, “Evaluation of a Treatment to Decrease the Risk of a Progressive Brain Disorder or to Slow Brain Aging” teaches methods and ways to use brain imaging to measure brain activity and/or structural changes to determine efficacy of putative treatments for brain-related disorders. Embodiments of the current invention can be used to improve and identify novel biomarkers and methods for the treatment and assessment of a variety of disease states that result in cognitive impairments, alterations, and/or deficiencies.

In order to develop or improve diagnosis, prognosis, and/or treatment of such disease states clinical trials and other studies must use cognitive testing to assess progression of the disease in order to determine whether the therapy under study has a positive effect on disease progression. However, the variability in patient response associated with cognitive testing, due to the progressive and variable course of the disease, is large enough to inhibit the ability of these tests to detect alteration in the status of an individual. The current methods can be used to detect and evaluate such alterations in the status of an individual.

### III. COMPUTER IMPLEMENTATION

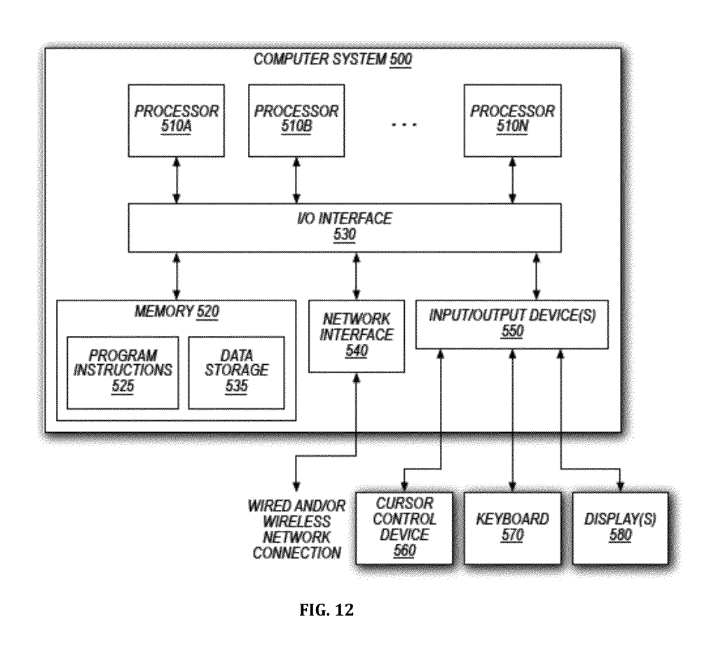

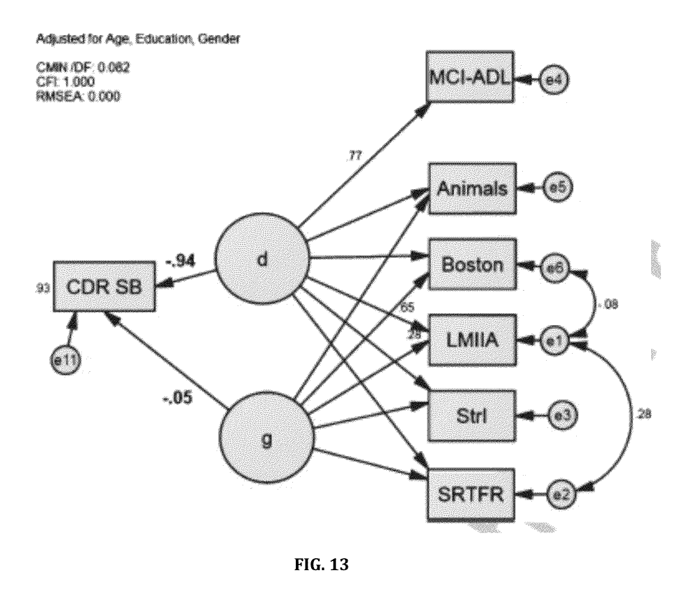

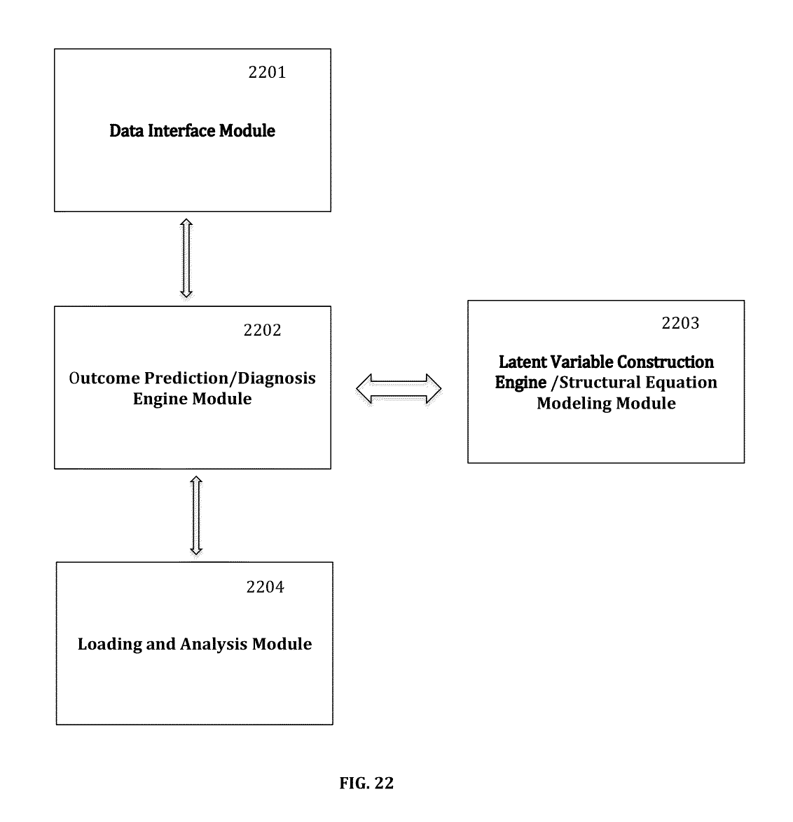

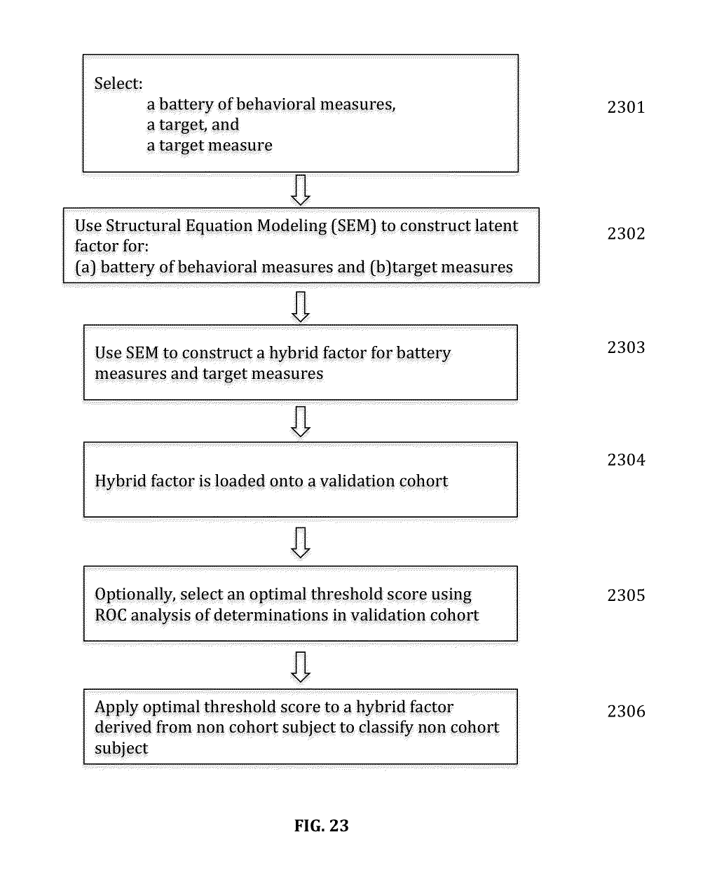

Embodiments of hybrid latent variable system may be implemented or executed by one or more computer systems. One such computer system is illustrated in FIG. 12. In various embodiments, computer system may be a server, a mainframe computer system, a workstation, a network computer, a desktop computer, a laptop, or the like. For example, in some cases, the system shown in FIG. 2, FIG. 22, FIG. 23 or the like may be implemented as computer system. Moreover, one or more of servers or devices may include one or more computers or computing devices generally in the form of a computer system. In different embodiments these various computer systems may be configured to communicate with each other in any suitable way, such as, for example, via a network.

As illustrated, the computer system includes one or more processors 510 coupled to a system memory 520 via an input/output (I/O) interface 530. Computer system 500 further includes a network interface 540 coupled to I/O interface 530, and one or more input/output devices 550, such as cursor control device 560, keyboard 570, and display(s) 580. In some embodiments, a given entity (e.g., hybrid latent variable system) may be implemented using a single instance of computer system 500, while in other embodiments multiple such systems, or multiple nodes making up computer system 500, may be configured to host different portions or instances of embodiments. For example, in an embodiment some elements may be implemented via one or more nodes of computer system 500 that are distinct from those nodes implementing other elements (e.g., a first computer system may implement an assessment of a hybrid latent variable assessment or system while another computer system may implement data gathering, scaling, classification etc.).

In various embodiments, computer system 500 may be a single-processor system including one processor 510, or a multi-processor system including two or more processors 510 (e.g., two, four, eight, or another suitable number). Processors 510 may be any processor capable of executing program instructions. For example, in various embodiments, processors 510 may be general-purpose or embedded processors implementing any of a variety of instruction set architectures (ISAs), such as the x86, POWERPC®, ARM®, SPARC®, or MIPS® ISAs, or any other suitable ISA. In multi-processor systems, each of processors 510 may commonly, but not necessarily, implement the same ISA. Also, in some embodiments, at least one processor 510 may be a graphics-processing unit (GPU) or other dedicated graphics-rendering device.

System memory 520 may be configured to store program instructions and/or data accessible by processor 510. In various embodiments, system memory 520 may be implemented using any suitable memory technology, such as static random access memory (SRAM), synchronous dynamic RAM (SDRAM), nonvolatile/Flash-type memory, or any other type of memory. As illustrated, program instructions and data implementing certain operations, such as, for example, those described herein, may be stored within system memory 520 as program instructions 525 and data storage 535, respectively. In other embodiments, program instructions and/or data may be received, sent or stored upon different types of computer-accessible media or on similar media separate from system memory 520 or computer system 500. Generally speaking, a computer-accessible medium may include any tangible storage media or memory media such as magnetic or optical media—e.g., disk or CD/DVD-ROM coupled to computer system 500 via I/O interface 530. Program instructions and data stored on a tangible computer-accessible medium in non-transitory form may further be transmitted by transmission media or signals such as electrical, electromagnetic, or digital signals, which may be conveyed via a communication medium such as a network and/or a wireless link, such as may be implemented via network interface 540.

In an embodiment, I/O interface 530 may be configured to coordinate I/O traffic between processor 510, system memory 520, and any peripheral devices in the device, including network interface 540 or other peripheral interfaces, such as input/output devices 550. In some embodiments, I/O interface 530 may perform any necessary protocol, timing or other data transformations to convert data signals from one component (e.g., system memory 520) into a format suitable for use by another component (e.g., processor 510). In some embodiments, I/O interface 530 may include support for devices attached through various types of peripheral buses, such as a variant of the Peripheral Component Interconnect (PCI) bus standard or the Universal Serial Bus (USB) standard, for example. In some embodiments, the function of I/O interface 530 may be split into two or more separate components, such as a north bridge and a south bridge, for example. In addition, in some embodiments some or all of the functionality of I/O interface 530, such as an interface to system memory 520, may be incorporated directly into processor 510.

Network interface 540 may be configured to allow data to be exchanged between computer system 500 and other devices attached to a network, such as other computer systems, or between nodes of computer system 500. In various embodiments, network interface 540 may support communication via wired or wireless general data networks, such as any suitable type of Ethernet network, for example; via telecommunications/telephony networks such as analog voice networks or digital fiber communications networks; via storage area networks such as Fiber Channel SANs, or via any other suitable type of network and/or protocol.

Input/output devices 550 may, in some embodiments, include one or more display terminals, keyboards, keypads, touch screens, scanning devices, voice or optical recognition devices, or any other devices suitable for entering or retrieving data by one or more computer system 500. Multiple input/output devices 550 may be present in computer system 500 or may be distributed on various nodes of computer system 500. In some embodiments, similar input/output devices may be separate from computer system 500 and may interact with one or more nodes of computer system 500 through a wired or wireless connection, such as over network interface 540.

As shown in FIG. 12, memory 520 may include program instructions 525, configured to implement certain embodiments described herein, and data storage 535, comprising various data accessible by program instructions 525. In an embodiment, program instructions 525 may include software elements of embodiments illustrated in FIG. 2, FIG. 22, FIG. 23 or the like. For example, program instructions 525 may be implemented in various embodiments using any desired programming language, scripting language, or combination of programming languages and/or scripting languages (e.g., C, C++, C#, JAVA®, JAVASCRIPT®, PERL®, etc). Data storage 535 may include data that may be used in these embodiments. In other embodiments, other or different software elements and data may be included.

A person of ordinary skill in the art will appreciate that computer system 500 is merely illustrative and is not intended to limit the scope of the disclosure described herein. In particular, the computer system and devices may include any combination of hardware or software that can perform the indicated operations. In addition, the operations performed by the illustrated components may, in some embodiments, be performed by fewer components or distributed across additional components. Similarly, in other embodiments, the operations of some of the illustrated components may not be performed and/or other additional operations may be available. Accordingly, systems and methods described herein may be implemented or executed with other computer system configurations.

### IV. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Validation of a Latent Variable Representing the Dementing Process

A. Results

Descriptive statistics are presented in Table 1. The TARCC baseline sample is relatively highly educated, and has a slight preponderance of females. The baseline data do not include cases with Mild Cognitive Impairment (MCI). The AD group is significantly older, less well educated, and more impaired relative to controls on multiple measures.

First, the inventors constructed a factor model of two latent variables: “g” and “f”. The latent construct g represents “Spearman's g”, i.e., a latent variable representing the shared variance across the observed cognitive performance variables. g explained 68.8% of the variance in observed psychometric performance. “f” represents a latent functional status factor derived from the eight observed IADL items and the six observed BADL items. The latent construct f explained 50.67% of the variance in observed variance in care-giver rated IADL/BADL.

The observed cognitive measures all loaded significantly on g (range: r=−0.65-−0.79; all p<0.001) (Table 2). LM II loaded most strongly (r=−0.79). Digit Span loaded least strongly (r=−0.65). The observed IADL/BADL items all loaded significantly on f (range: r=−0.37-−0.84; all p<0.001) (Table 2). Shopping and responsibility for medication adherence loaded most strongly (both r=−0.84). Toileting loaded least strongly (r=−0.37).

In a multivariate regression (Model 1; FIG. 1), g and f were each strong significant and independent predictors of CDR SOB. Together, g and f explained 86% of the variance in CDR scores. Nonetheless, the model did not fit adequately well (Table 2). Significant inter-correlations amongst the residuals (not shown in FIG. 1), support the existence of an additional latent variable.

TABLE 1
Descriptive Statistics

| | | AD | Controls | | |
|---|---|---|---|---|---|
| | | N = 605 | N = 350 | Total | |
| | Variable N | Mean (SD) | Mean (SD) | Sample | p |
| Gender (% female) | 955 | 59 | 65 | 61 | 0.07 |
| Age at Visit | 955 | 76.6 (8.3) | 71.0 (8.7) | 74.5 (8.86) | <0.001 |
| Education | 955 | 14.3 (3.2) | 15.4 (2.7) | 14.7 (3.0) | <0.001 |
| MMSE | 955 | 20.3 (5.5) | 29.3 (0.9) | 23.6 (6.2) | <0.001 |
| CDR (Sum of Boxes) | 949 | 6.6 (3.7) | 0.0 (0.1) | 4.2 (4.4) | <0.001 |
| GDS (30 item) | 675 | 5.0 (4.7) | 2.8 (2.9) | 4.0 (4.2) | <0.001 |
| COWA | 902 | 7.2 (3.4) | 11.3 (2.9) | 8.8 (3.8) | <0.001 |
| Boston Naming Test | 927 | 6.4 (3.6) | 12.4 (3.1) | 8.7 (4.5) | <0.001 |
| WMS LM II | 714 | 3.5 (2.0) | 13.7 (2.8) | 7.6 (5.5) | <0.001 |
| WMS VR II | 409 | 4.0 (2.3) | 13.6 (3.1) | 9.8 (5.5) | <0.001 |
| DST | 802 | 8.3 (3.0) | 11.7 (2.9) | 9.5 (3.4) | <0.001 |
| IADL (Summed) | 440 | 15.7 (6.3) | 7.8 (1.0) | 11.3 (5.8) | <0.001 |
| Complete Cases | 335 | | | | |

CDR = Clinical Dementia Rating scale;
COWA = Controlled Oral Word Association Test;
DST = Digit Span Test;
GDS = Geriatric Depression Scale;
IADL = Instrumental Activities of Daily Living;
MMSE = Mini-mental State Exam;
SD = standard deviation;
WMS LM II = Weschler Memory Scale: Delayed Logical Memory;
WMS VR II = Weschler Memory Scale: Delayed Visual Reproduction.

TABLE 2
Selected Model 1 Parameters

| | Factor | β | S.E. | p |
|---|---|---|---|---|
| Boston Naming Test | g | −0.78 | 0.13 | <0.001 |
| COWA | g | −0.72 | 0.12 | <0.001 |
| DST | g | −0.65 | 0.13 | <0.001 |
| WMS LM II | g | −0.79 | 0.17 | <0.001 |
| WMS VR II | g | −0.76 | 0.20 | <0.001 |
| IADL1 (telephone) | f | −0.80 | 0.31 | <0.001 |
| IADL2 (shopping) | f | −0.84 | 0.33 | <0.001 |
| IADL3 (cooking) | f | −0.74 | 0.43 | <0.001 |
| IADL4 (housekeeping) | f | −0.75 | 0.38 | <0.001 |
| IADL5 (laundry) | f | −0.61 | 0.30 | <0.001 |
| IADL6 (transportation) | f | −0.76 | 0.47 | <0.001 |
| IADL7(finances) | f | −0.82 | 0.30 | <0.001 |
| IADL8 (medications) | f | −0.84 | 0.27 | <0.001 |
| ADL1 (toileting) | f | −0.37 | 0.02 | <0.001 |
| ADL2 (eating) | f | −0.47 | 0.01 | <0.001 |
| ADL3 (dressing) | f | −0.63 | 0.02 | <0.001 |
| ADL4 (grooming) | f | −0.68 | 0.02 | <0.001 |
| ADL5 (ambulation) | f | −0.63 | 0.02 | <0.001 |
| ADL6 (bathing) | f | −0.61 | 0.02 | <0.001 |
| CDR (Sum of Boxes) | g | 0.55 | 0.09 | <0.001 |
| CDR (Sum of Boxes) | f | −0.64 | 0.08 | <0.001 |

Fit Indices

| | |
|---|---|
| χ²/DF | 6.42, p < 0.001 |
| CFI | 0.903 |
| RMSEA | 0.075 |

ADL = Basic Activities of Daily Living;
CDR = Clinical Dementia Rating scale;
CFI = Corrected Fit Index;
COWA = Controlled Oral Word Association Test;
DF = degrees of freedom;
DST = Digit Span Test;
IADL = Instrumental Activities of Daily Living;
RMSEA = Root Mean Square Error of Association;
S.E. = Standard Error;
WMS LM II = Weschler Memory Scale: Delayed Logical Memory;
WMS VR II = Weschler Memory Scale: Delayed Visual Reproduction.

Next, the inventors introduced a third latent variable “δ” or “d” (Base Model 2a, Table 3). Model 2's design (FIG. 2) suggests that δ, g′ and f are orthogonal to each other. The inventors confirmed this by correlating each with the other two. No correlations were significant (data not shown). The latent construct δ represents the variance shared between cognitive and IADL/BADL measures [i.e., any and all dementing process(es) afflicting the sample]. The creation of δ attenuated the association between g and several measures of cognitive performance (range r=0.32-0.48; all p<0.001). The inventors relabeled g as “g′” to acknowledge this effect. Together, g′ and δ accounted for 59.6% of the variance in our cognitive battery. The latent construct δ accounted for 37.2% independently of g′. The remainder was attributable to residual “measurement error”.

The latent construct f was also affected by the creation of δ. The latent construct f retained relatively strong associations with the BADL items (range r=0.35-0.62, all p<0.001) but lost its formerly strong associations with IADL items (range r=0.10-0.28), one of which (cooking) no longer loaded significantly on f (r=0.10, p=0.068). This confirms our expectation that IADL items are more relevant to dementing illness (through 6) than are BADL items.

The latent construct δ was significantly and inversely associated with each cognitive performance measure (range: r=−0.55-−0.67; all p<0.001) (Table 3). It was most strongly associated with WMS VRII (r=−0.67), and least strongly associated with DST (r=−0.55).

TABLE 3
Selected Base Model 2a Parameters

| | Factor | β | S.E. | p |
|---|---|---|---|---|
| Boston Naming Test | g′ | 0.47 | 0.19 | <0.001 |
| COWA | g′ | 0.48 | 0.17 | <0.001 |
| DST | g′ | 0.32 | 0.25 | <0.001 |
| WMS LM II | g′ | 0.52 | 0.24 | <0.001 |
| WMS VR II | g′ | 0.41 | 0.25 | <0.001 |
| IADL1 (telephone) | f | 0.19 | 0.05 | <0.001 |
| IADL2 (shopping) | f | 0.11 | 0.05 | 0.04 |
| IADL3 (cooking) | f | 0.10 | 0.07 | 0.07 |
| IADL4 (housekeeping) | f | 0.22 | 0.06 | <0.001 |
| IADL5 (laundry) | f | 0.28 | 0.04 | <0.001 |
| IADL6 (transportation) | f | 0.13 | 0.07 | 0.01 |
| IADL7(finances) | f | 0.11 | 0.05 | 0.03 |
| IADL8 (medications) | f | 0.17 | 0.04 | <0.001 |
| ADL1 (toileting) | f | .044 | 0.03 | <0.001 |
| ADL2 (eating) | f | 0.35 | 0.01 | <0.001 |
| ADL3 (dressing) | f | 0.49 | 0.02 | <0.001 |
| ADL4 (grooming) | f | 0.48 | 0.03 | <0.001 |
| ADL5 (ambulation) | f | 0.42 | 0.03 | <0.001 |
| ADL6 (bathing) | f | 0.62 | 0.02 | <0.001 |
| Boston Naming Test | δ | −0.61 | 0.14 | <0.001 |
| COWA | δ | −0.54 | 0.13 | <0.001 |
| DST | δ | −0.55 | 0.12 | <0.001 |
| WMS LM II | δ | −0.66 | 0.18 | <0.001 |
| WMS VR II | δ | −0.67 | 0.20 | <0.001 |
| IADL1 (telephone) | δ | 0.79 | 0.03 | <0.001 |
| IADL2 (shopping) | δ | 0.87 | 0.03 | <0.001 |
| IADL3 (cooking) | δ | 0.76 | 0.04 | <0.001 |
| IADL4 (housekeeping) | δ | 0.72 | 0.04 | <0.001 |
| IADL5 (laundry) | δ | 0.51 | 0.03 | <0.001 |
| IADL6 (transportation) | δ | 0.76 | 0.05 | <0.001 |
| IADL7(finances) | δ | 0.83 | 0.03 | <0.001 |
| IADL8 (medications) | δ | 0.83 | 0.03 | <0.001 |
| ADL1 (toileting) | δ | 0.25 | 0.02 | <0.001 |
| ADL2 (eating) | δ | 0.40 | 0.01 | <0.001 |
| ADL3 (dressing) | δ | 0.51 | 0.02 | <0.001 |
| ADL4 (grooming) | δ | 0.56 | 0.02 | <0.001 |
| ADL5 (ambulation) | δ | 0.51 | 0.02 | <0.001 |
| ADL6 (bathing) | δ | 0.46 | 0.02 | <0.001 |
| CDR (Sum of Boxes) | g | −0.18 | 0.01 | <0.001 |
| CDR (Sum of Boxes) | f | 0.22 | 0.17 | <0.001 |
| CDR (Sum of Boxes) | δ | 0.84 | 0.12 | <0.001 |

Fit Indices

| | |
|---|---|
| χ²/DF | 1.54, p < 0.001 |
| CFI | 0.992 |
| RMSEA | 0.024 |

ADL = Basic Activities of Daily Living;
CDR = Clinical Dementia Rating scale;
CFI = Corrected Fit Index;
COWA = Controlled Oral Word Association Test;
DF = degrees of freedom;
DST = Digit Span Test;
IADL = Instrumental Activities of Daily Living;
RMSEA = Root Mean Square Error of Association;
S.E. = Standard Error;
WMS LM II = Weschler Memory Scale: Delayed Logical Memory;
WMS VR II = Weschler Memory Scale: Delayed Visual Reproduction.

The latent construct δ was also strongly and positively associated with each IADL item (range: r=0.51-0.87). The latent construct 6 was most strongly associated with shopping (r=0.87) and least strongly associated with laundry (r=0.51). Each BADL item loaded significantly (and positively) on δ, but the strength of these associations was relatively weak (range: r=0.25-0.56). The latent construct 6 was most strongly associated with ADL4 (grooming) (r=0.56) and least strongly associated with ADL 1 (toileting) (r=0.25). Thus, in contrast to f in Model 1, δ appears to be relatively specifically related to variance in IADL and not BADL items.

The latent construct δ is intended to specifically reflect the effect of dementing process(es) within a cohort. As a test of δ's construct validity, the inventors regressed the base model of g′, δ and f onto CDR SOB (Table 4: Model 2a, FIG. 2). Together, g′, f and δ explained 90% of the variance in CDR SOB. However, this was almost entirely mediated by δ (r=0.84; p<0.001). In contrast to Model 1, g's association was severely attenuated, but remained significant (r=−0.18; p=<0.001). The latent construct f's former association with dementia severity was also attenuated (partial r=0.22; p<0.001).

TABLE 4
Regression Model Parameters

| | | | |
|---|---|---|---|
| Factor | β | S.E. | p |

Model 2a

| | | | | |
|---|---|---|---|---|
| CDR (Sum of Boxes) | g′ | −0.18 | 0.10 | <0.001 |
| CDR (Sum of Boxes) | f | 0.22 | 0.17 | <0.001 |
| CDR (Sum of Boxes) | δ | 0.84 | 0.12 | <0.001 |

Model 2b

| | | | | |
|---|---|---|---|---|
| MMSE | g′ | −0.31 | 0.17 | <0.001 |
| MMSE | f | −0.06 | 0.22 | 0.27 |
| MMSE | δ | −0.82 | 0.17 | <0.001 |

Model 2c

| | | | | |
|---|---|---|---|---|
| GDS | g′ | −0.17 | 0.22 | 0.001 |
| GDS | f | −0.04 | 0.23 | 0.20 |
| GDS | δ | 0.18 | 0.19 | <0.001 |

CDR = Clinical Dementia Rating scale (sum of boxes);
GDS = Geriatric Depression Scale (30 items);
MMSE = Mini-mental Status Examination;
S.E. = Standard Error.

Discriminant validity is provided by multivariate regression models of MMSE and GDS scores (Table 4; Models 2b-c). The MMSE is a measure of global cognition and should be more strongly associated with a dementing process than the GDS, a measure of depressed mood. As expected, δ's association with these measures was weakened relative to that with CDR SOB. g's association with MMSE scores (Model 2b) was strengthened relative to that with CDR SOB. g′ and δ were weakly associated with GDS scores (Model 2c). The latent construct f did not contribute significantly to either of those outcomes (FIG. 2).

Table 5 presents the results of an ROC analysis. The latent variables g′, f, and δ were tested as independent predictors of TARCC consensus clinical diagnoses (i.e., “AD” vs. “control”). The latent construct δ achieved the most accurate discrimination (AUC=0.942) (Table 5, FIG. 3). The latent construct g′ (AUC=0.790) was more accurate in this discrimination than was f (AUC=0.550). When CDR scores were dichotomized about a threshold of 1.0, δ again achieved the most accurate discrimination (AUC=0.996) (data not shown).

TABLE 5
ROC Analysis of g′ f and d as Predictors of
Adjudicated Clinical Dementia Status - Area Under the Curve

| Test Result Variable(s) | Area |
|---|---|
| δ | 0.942 |
| f | 0.550 |
| g′ | 0.790 |

a. Under the nonparametric assumption;
b. Null hypothesis: true area = 0.5

B. Methods:

Subjects:

These data represent baseline data from the TARCC cohort's first wave (circa 2008-2009). Subjects included N=955 TARCC participants (605 AD cases, 350 controls). The methodology of the TARCC project has been described in detail elsewhere (Waring et al., 2008). Each participant underwent a standardized annual examination at the respective site that includes a medical evaluation, neuropsychological testing, and clinical interview. Diagnosis of AD status was based on National Institute for Neurological Communicative Disorders and Stroke—Alzheimer's Disease and Related Disorders Association (NINCDS—ADRDA) criteria (McKhann et al., 1984). Controls performed within normal limits on their psychometric assessments. Institutional Review Board approval was obtained at each site and written informed consent was obtained for all participants.

Clinical Variables:

Instrumental Activities of Daily Living (IADL) were assessed using care-giver ratings (Lawton and Brody (1969) Gerontologist 9, 179-186). The ability to use the telephone (IADL1), shopping (IADL2), food preparation (IADL3), housekeeping (IADL4), laundry (IALD5) use of transportation (IALD6) ability to handle finances (IALD7) and responsibility for medication adherence (IALD8) were each rated on a Likert scale ranging from 0 (no impairment) to 3 (specific incapacity).

Basic Activities of Daily Living (BADL) were assessed using the care-giver rated Physical Self-Maintenance Scale (PSMS) (Lawton and Brody (1969) Gerontologist 9, 179-186). The subject's ability to toilet (ADL1), eat (ADL2), dress (ADL3), groom (ADL4), ambulate (ADL5), and bathe (ADL6) were each rated on a Likert scale ranging from 0 (no impairment) to 3 (specific incapacity).

Cognitive Battery

Executive Control Function Measures:

The Controlled Oral Word Association (COWA) (Benton and Hamsher (1989) Multilingual Aphasia Examination. Iowa City, Iowa: AJA Associates) is a test of oral word production (verbal fluency). The patient is asked to say as many words as they can, beginning with a certain letter of the alphabet. Reduced word fluency scores are associated with frontal lobe impairment, particularly in the left hemisphere (Baldo et al. (2001) J Int Neurosci 7, 586-596; Stuss et al. (1998) J Int Neurosci 4, 265-278).

Memory:

Logical Memory II (Wechsler (1997) Wechsler Memory Scale—Third Edition. San Antonio, Tex.: The Psychological Corporation): Following a thirty minute delay, the subject recalls two paragraphs read aloud. Delayed paragraph recall has been useful clinically in identifying dementia and tracking progression of the disease.

Attention:

Digit Span Test (DST) (Wechsler (1997) Wechsler Memory Scale—Third Edition. San Antonio, Tex.: The Psychological Corporation): Digit span sums the longest set of numbers the subject can repeat back in correct order (forwards and backwards) immediately after presentation on 50% of trials.

Verbal:

The Boston Naming Test (BOSTON) (Kaplan et al. (1983) The Boston Naming Test. Experimental edition. Boston: Kaplan & Goodglass. 2nd ed., Philadelphia: Lea & Febiger): This is a confrontation naming test that requires the subject to verbally name each of 60 line drawings of objects of increasingly low frequency.

Non-Verbal:

WMS Visual Reproduction II (Wechsler (1997) Wechsler Memory Scale—Third Edition. San Antonio, Tex.: The Psychological Corporation): The subject is asked to reproduce five, simple to complex figures following a thirty minute delay.

Outcome Measures

The Clinical Dementia Rating Scale (CDR) Sum of Boxes (SOB)

(Hughes et al. (1982) Br J Psychiatry 140, 566-572): The CDR is used to evaluate dementia severity. The rating assesses the patient's cognitive ability to function in six domains—memory, orientation, judgment and problem solving, community affairs, home and hobbies and personal care. Information is collected during an interview with the patient's caregiver. Optimal SOB ranges corresponding to global CDR scores are 0.5-4.0, for a global score of 0.5, 4.5-9.0, for a global score of 1.0, 9.5-15.5, for a global score of 2.0, and 16.0-18.0, for a global score of 3.0 (O'Bryant et al. (2008) Arch Neurol 65, 1091-95).

The MMSE (Folstein et al. (1975) J Psychiatr Res 12, 189-98) is a well known and widely used test for screening cognitive impairment (Tombaugh and McIntyre (1992) J Am Geriatrics Soc 40, 922-35). Scores range from 0 to 30. Scores less than 24 reflect cognitive impairment.

The Geriatric Dementia Rating Scale (GDS):

Depressive symptoms were assessed using the short Geriatric Depression Scale (GDS) (Sheikh and Yesavage (1986) Clin Gerontologist 5, 165-73; Maxiner et al., 1995). GDS scores range from zero-30. Higher scores are worse. A cut-point of 9-10 best discriminates clinically depressed from non-depressed elderly.

Statistical Analyses

Statistical analysis was performed using Analysis of Moment Structures (AMOS) software (Arbuckle (2006) Analysis of Moment Structures-AMOS (Version 7.0) [Computer Program]. Chicago: SPSS). Latent variables of interest were constructed from confirmatory factor analyses performed in a structural equation framework. Residual covariances were explicitly estimated for each observed measure. All observed measures, latent indicators and outcomes, were adjusted for age, gender and education. The latent variables of interest were validated as predictors of observed TARCC outcomes in multivariate regression models and by Receiver Operating Characteristic (ROC) analyses. Three multivariate regression models were developed using the latent variables as simultaneous predictors of SOB, MMSE and GDS scores. In ROC analyses, latent variables were used to predict TARCC adjudicated dementia status (AD case vs. control), or CDR score≧1.0.

Missing Data:

Some variables (e.g., VRII) were not used at all sites in TARCC's first wave. However, only the ROC analyses were limited to complete cases. Elsewhere, Full Information Maximum Likelihood (FIML) methods were used to address missing data. FIML uses the entire observed data matrix to estimate parameters with missing data. In contrast to listwise or pairwise deletion, FIML yields unbiased parameter estimates, preserves the overall power of the analysis, and is arguably superior to alternative methods, e.g., multiple imputation (Schafer and Graham (2002) Psychol Methods, 7, 147-77; Graham (2009) Ann Rev Psychol 6, 549-76).

Fit Indices:

The validity of structural models was assessed using two common test statistics. A non-significant chi-square signifies that the data are consistent with the model (Bollen and Long (1993) Testing Structural Equation Models. Sage Publications, Thousand Oaks, Calif.). The comparative fit index (CFI), with values ranging between 0 and 1, compares the specified model with a model of no change (Bentler (1990) Psychol Bull 107, 238-46). CFI values below 0.95 suggest model misspecification. Values of 0.95 or greater indicate adequate to excellent fit. A root mean square error of approximation (RMSEA) of 0.05 or less indicates a close fit to the data, with models below 0.05 considered “good” fit, and up to 0.08 as “acceptable” (Browne and Cudeck (1993) Alternative ways of assessing model fit, in Bollen, K. A., Long, J. S. (Eds.), Testing structural equation models Sage Publications, Thousand Oaks, Calif., pp. 136-62). All three fit statistics should be simultaneously considered to assess the adequacy of the models to the data.

ROC Curves:

The diagnostic performance or accuracy of a test to discriminate diseased from normal cases can be evaluated using ROC curve analysis (Metz (1978) Sem Nuc Med 8, 283-98; Zweig and Campbell (1993) Clin Chem 39, 561-77). Briefly the true positive rate (Sensitivity) is plotted as a function of the false positive rate (100-Specificity) for different cut-off points of a parameter. Each point on the ROC curve represents a sensitivity/specificity pairing corresponding to a particular decision threshold. The area under the ROC curve (AUC) is a measure of how well a parameter can distinguish between two diagnostic groups (diseased/normal). The analysis was performed in Statistical Package for the Social Sciences (SPSS) (2009).

### Example 2

### Testing a New Model of Dementing Processes in Non-Demented Persons

The inventors have studied 547 well elderly retirees as part of the Air Force Villages' (AFV) Freedom House Study (FHS). The AFV is a 1500-bed CCRC in San Antonio, Tex. that is open to Air Force officers and their dependents. At baseline, the FHS subjects represented a random sample of AFV residents over the age of 70 years living at non-institutionalized levels of care. Informed consent was obtained prior to their evaluations.

A subset of FHS participants (n=187) were administered a formal neuropsychological test battery that included standardized tests of memory, language, and ECF. This subgroup was slightly older at baseline than the larger FHS cohort (mean age of 79.0 years vs. 77.7 years, respectively), but did not differ significantly with regard to gender, education, baseline level of care, or Mini-Mental State Examination (MMSE) scores.

At baseline, the cohort is cognitively normal for age, relatively highly functioning and non-institutionalized. The baseline mean and variability about that mean for each cognitive measure is available elsewhere (Royall et al., 2005a; b). We have also demonstrated that there is significant variability with regard to the cohort's longitudinal rates of change in cognitive performance over time. These changes are clearly related to concurrent declines in functional status. Thus, despite the fact that the cohort was non-demented at baseline, it is demonstrably suffering from a dementing process that is capable of disabling it in time.

The inventors first built a factor model of a latent variable, “g”, representing the variance shared across a cognitive measures battery. Each measure loaded significantly on g. g was most strongly loaded by WAIS-R SIM (r=0.67), COWA (r=0.62) and VOCAB (r=0.62), and least strongly loaded by WAIS-R BLOCK and DSS (both r=0.50). All loadings were significant (p<0.001).

Next the inventors correlated FSI with g. “Functional Status” was significantly associated with g (r=−0.41), which explained 16.8% of its variance. Thus, functional status shared a small but significant fraction of the variance in cognitive performance (i.e., g).

g explained 52.3% of the cognitive battery's variance, but exhibited marginally acceptable fit (χ²: F=67.5; df 18, p<0.001; RMSEA=0.070; BCC=163.38). Moreover, significant correlations amongst the residuals (data not shown) suggested that a multifactorial model might better fit these data. Therefore, the inventors constructed a second factor, “δ”, representing the shared variance between our FSI and cognitive performance. Unlike the model in FIG. 1, this model uses Functional status as an indicator of a latent variable rather than its correlate. This effectively parses the shared variance across the cognitive measures (i.e., g) into a larger fraction that is not related to functional status (i.e., g′), and a smaller fraction that is (i.e., δ). This two factor model provides better fit to the data than the one factor model represented in FIG. 1 (χ²: F=32.5; df 17, p=0.01; RMSEA=0.040; BCC=155.08).

δ is significantly related to “Functional Status”, (r=0.35), and negatively related to cognitive performance. All loadings on δ are significant. In contrast to g and g′, δ is most strongly loaded by DSS (r=−0.67). WAIS-R BLOCK's association with g′ was attenuated, and the loadings of the CVLT and WAIS-R DSS on g′ are no longer significant after the creation of “δ”.

Next the inventors examined the clinical significance of δ vs. g′ in multivariate regression models of a variety of clinical outcomes. After adjusting for age, education and gender, g and δ were independently, significantly and moderately associated with DRS:MEM, MMSE, and EXIT25 scores. δ alone was moderately associated with baseline Trails B scores, and strongly associated with Trails A. Neither construct was significantly associated with baseline level of care (restricted variability), nor with 5-year prospective all-cause mortality.

Finally, the inventors examined g′ and δ as independent predictors of 3-year prospective change in cognitive performance, in multivariate regression models of linear longitudinal change derived from LGC models, adjusted for age, education, and gender. All models showed excellent fit (i.e., RMSEA<0.05) except ΔCLOX2, which was acceptable (RMSEA=0.052). Once again, δ was most strongly associated with non-verbal measures (DSS; r=−0.75) while g′ was most strongly associated with verbal measures (VOCAB (r=0.62).

δ was significantly correlated with ΔCLOX2, ΔEXIT25, ΔDRS:MEM, ΔTrails A and ΔTrails B. ΔMMSE showed a trend. g′ was not significantly associated with prospective change in any clinical outcome independently of δ.

The inventors have performed additional analyses of this latent variable (d)'s properties. The latent variable d exhibits factor equivalence across similar samples. “Factor equivalence” refers to the statistical equivalence of a latent variable when constructed in two difference samples. The inventors confirmed this by randomly splitting TARCC's cohort into two approximately equal groups of n=1018 and n=999 respectively. There was no significant difference between d's model's fit when comparing the original to one where the associations between d and each of its indicators were constrained to be equal across those two groups [i.e., χ2=23.7 (14); p<0.05 vs. χ2=29.8 (20); p<0.05; p>0.05 by Chi Sq table)]. Thus, the latent variable d exhibits factor equivalence across similar samples. This finding suggests that d scores developed in one population (i.e., the validation sample) will be generalizable to any similar population (i.e., the population targeted for diagnosis through d scores).

The inventors have also constructed a d homolog from longitudinally measured cognitive and functional performance (i.e, IADL scores). In this application, d and g were constructed in each of four annually collected waves of data from TARCC. The resulting latent endophenotypes were then used as indicators of two latent growth curve models of change in d and g respsectively. This resulted in four new latent variables, d and g's estimated baseline values (i.e, d and g′) and their estimated slopes (Δd and Δg′).

The DIGIT, COWA, BOSTON and IADL showed significant declines over time while Logical Memory (LMII) and Visual Recall (VRII) demonstrated significant increases [χ2=1152 (df=229); CFI=0.968; RMSEA=0.043]. All indicator loadings were significant for the four latent variables: g′, Δg′, d and Δd, yielding four distinct factors. This model demonstrated good fit to the data [χ2=543 (df=245); CFI=0.991; RMSEA=0.023]. After adjustment for demographic covariates and baseline CDR sum of boxes (CDR-SB) scores, d and Δd were significantly independently associated with CDR-SB at wave 4, explaining 25% and 49% of its variance, respectively. The latent variable g′ significantly explained 3% of CDR-SB4 variance independently of d and Δd. Δg′ was not significantly associated with CDR-SB4. Baseline CDR-SB explained 16% of CDR-SB4 variance, independently of d, Δd and g′. Thus, the latent hybrid variables generated by our method are not only strong predictors of an individual's current clinical diagnosis or status, but they can be used in second order analytical processes to estimate their rates of change in time, and those slopes are independently associated with future clinical states (Abstract #39733: Palmer and Royall, (2013) Future Dementia Status is Almost Entirely Explained by the Latent Variable “d”'s Baseline and Change. 2013 Alzheimer's Association International Conference (AAIC). Boston, Mass.; manuscript in press).

### Example 3

### Association of the Default Mode Network with Depressive Symptom-Related Cognitive Changes

It has been suggested that depressive symptoms in Alzheimer's disease (AD) may reflect a specific syndrome of depression in AD (Vilalta-Franch et al. (2006) Am J Geriatr Psychiatry 14, 589-97). Depression in non demented persons has been identified as a possible risk factor for incident AD (Speck et al. (1995) Epidemiol 6, 366-69; Green et al. (2003) Arch Neurol 60, 753-59; Steenland et al. (2012) J Alzheimers Dis 31, 265-75). In a recent meta-analysis, depression appeared to double the risk of AD (Ownby et al. (2006) Arch Gen Psychiatry 63, 530-38). Depressive symptoms are common in mild cognitive impairment (MCI) (Lee et al. (2007) Int Psychogeriatr 19, 125-35) and appear to hasten conversion from MCI to clinical AD (Gabryelewicz et al. (2007) Int J Geriatr Psychiatry 22, 563-67). Even subclinical depressive symptoms may be sufficient to convey this risk (Rosenberg (2012) Am J Geriatr Psychiatry, doi:10.1097/JGP.0b013e318252e41a).

The mechanism(s) by which depression and depressive symptoms might affect these risks has not been well established. However, it has been shown that depressive symptoms are associated with incident changes in executive control, not memory (Royall et al. (2012) Int J Geriatr Psychiatry 27, 89-96), and that depressive symptom-related cognitive change is not mediated through AD pathology (Royall and Palmer (2012) Alzheimers Dement doi:pii:S1552-5260(12)00022-2. 10.1016/j jalz.2011.11.009). Similarly, a history of past depressive episodes is not associated with the distribution of (11)C Pittsburgh Compound B (PiB) binding (Madsen et al. (2012) Neurobiol Aging 33, 2334-42) and depressive symptoms are not associated with apolipoprotein E4 (ApoE4) in MCI (Nose et al. (2012) Int J Geriatr Psychiatry, doi:10.1002/gps.3803). These findings suggest that depression's effects on dementia status are independent of the AD process. Therefore, depression may itself be dementing. Moreover, because none of these studies involved clinically depressed persons, sub-syndromal depressive symptoms may indicate an independent dementing process.

The inventors use structural equation modeling (SEM) to explicitly distinguish dementia relevant variance in cognitive task performance (i.e., δ) from that which is unrelated to a dementing process (i.e., g′) (Royall and Palmer (2012) J Neuropsychiatry Clin Neurosci 24, 37-46; Royall et al. (2012) J Alzheimers Dis 30, 639-49). Together, g′ and δ effectively comprise Spearman's g (i.e., general intelligence) (Spearman (1904) Am J Psychol 15, 201-93). The inventors recently validated δ in the Texas Alzheimer's Research and Care Consortium (TARCC), a well characterized cohort of AD cases and controls (Royall et al. (2012) J Alzheimers Dis 30, 639-49).

One of the advantages of this approach is that δ's factor scores represent a continuously varying and arguably measurement error free dementia endophenotype. Biomarkers of this endophenotype have been examined, and recently co-localized δ specifically with grey matter atrophy in the Default Mode Network (DMN) (Royall et al. (2012) J Alzheimers Dis 32, 467-78). The DMN is comprised of highly interconnected neocortical regions that are active during wakeful self-reflection and introspection, and inactive during task specific processing (Uddin et al. (2009) Hum Brain Mapp 30, 625-37). Its hubs include parts of the medial temporal lobe, the medial prefrontal cortex, the posterior cingulate, the precuneus, and themedial, lateral, and inferior parietal cortex (Buckner et al. (2008) Ann NY Acad Sci 1124, 1-38). The DMN is abnormal in AD, but also in depression (Sheline et al. (2009) Proc Natl Acad Sci USA 106, 1942-47). Depression-related atrophy in DMN related regions (Goveas et al. (2011) J Affect Disord 132, 275-84) may provide an explanation for the disabling, and therefore intrinsically “dementing” nature of depressive illness.

In this analysis, the inventors return to the same dataset (i.e., the Brain Aging Project (BAP) of the University of Kansas Department of Neurology's Alzheimer's Disease Center) to examine whether there is overlap between the cognitive correlates of depressive symptoms (DEPCOG) and the cognitive correlates of functional status (i.e., δ), and whether DEPCOG can also be specifically associated with structural changes in the DMN. If so, then sub-syndromal depression itself, independent of AD lesions, may be responsible for some cases of incident clinical “AD”, suggesting new opportunities for the latter's diagnosis, prevention, and treatment.

A. Results

Sample demographics are presented in Table 6. Clinical assessment means are presented in Table 7. All groups had sub-clinical mean GDSs and GDSc scores. However, there were significant cross-group differences by both measures (by MANOVA, adjusted for age, education, and gender). MCI cases exhibited significantly more depressive symptoms by both measures than either AD or controls. There were no significant differences between AD cases and controls on either measure. Although AD and MCI cases were significantly more likely to report a past history of depression than controls, MCI cases were no less likely to report a past history of depression than AD cases. There were no group differences with regard to the current use of either benzodiazepines or serotonin-selective reuptake inhibitors. AD cases were more likely than either MCI cases or controls to use other psychotropics (all group comparisons by post hoc Honest Significant Difference test for unequal n).

First, δ was replicated from a more circumscribed cognitive and clinical assessment (FIG. 13). As in previous analyses, the new latent construct “d” was a strong independent predictor of CDR-SB (r=−0.94, p<0.001), and was more strongly labeled by IADL (i.e., MCI-ADL; r=0.77, p<0.001) than by any cognitive measure (range r=0.52, Animals; −0.75, SRTFR; all p<0.001). This model had excellent fit (Table 8).

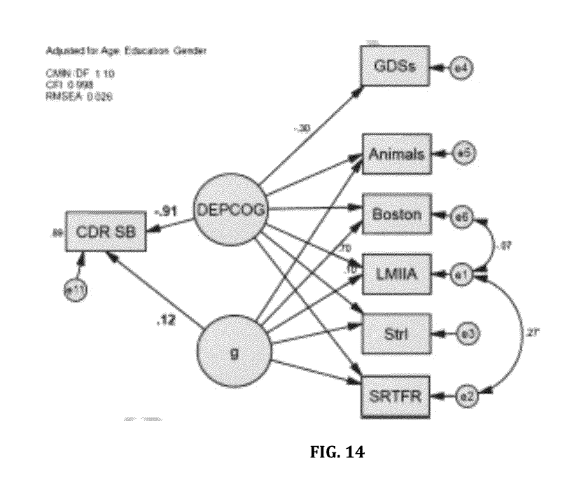

Next, DEPCOG was constructed from the same cognitive battery (FIG. 14). DEPCOG represents the shared variance between these cognitive measures and self-rated GDS scores. Aside from the fact that MCI ADL has been replaced by GDSs, Model 2 is identical to Model 1.

TABLE 6
Subject characteristics

| | | MCI mean | AD mean | Total mean |
|---|---|---|---|---|
| | Control mean | (SD) | (SD) | (SD) |
| Variable | (SD) (n = 76) | (n = 47) | (n = 23) | (n = 146) |
| Age | 74.2 (7.2) | 75.9 (6.5) | 73.2 (5.8) | 74.6 (6.8) |
| (years) | | | | |
| % Female | 58% | 62% | 70% | 61% |
| Education | 16.3 (2.7) | 14.9 (3.2) | 15.2 (3.0) | 15.7 (3.0) |
| (years) | | | | |
| Mini- | 29.4 (0.8) | 28.1 (1.3) | 22.1 (3.1) | 27.8 (3.0) |
| Mental | | | | |
| State | | | | |
| Exam | | | | |
| Clinical | 0 (0.1) | 2.7 (1.1) | 4.2 (1.2) | 1.5 (1.8) |
| Dementia | | | | |
| Rating | | | | |
| scale | | | | |
| Sum of | | | | |
| Boxes | | | | |

TABLE 7
Raw clinical means

| | Control | | | |
|---|---|---|---|---|
| | mean | MCI mean | AD mean | Total mean |
| | (SD) | (SD) | (SD) | (SD) |
| Variable | (n = 76) | (n = 47) | (n = 23) | (n = 146) |
| Boston naming | 14.2 (1.0) | 12.7 (2.7) | 9.4 (3.6) | 13.0 (2.8) |
| test (15 item) | | | | |
| LMIIA | 10.8 (4.6) | 4.6 (4.8) | 1.1 (2.0) | 7.3 (5.8) |
| SRTFR | 28.3 (6.3) | 17.5 (8.8) | 6.0 (5.0) | 21.3 (10.8) |
| Category | 18.6 (4.3) | 15.5 (4.5) | 9.2 (3.9) | 16.1 (5.4) |
| fluency: | | | | |
| animals | | | | |
| StrI | 35.9 (8.7) | 27.0 (7.8) | 14.5 (8.8) | 29.7 (11.4) |
| MCI-ADL | 49.4 (2.3) | 43.1 (5.4) | 36.4 (10.2) | 45.1 (7.1) |
| GDS subject | 0.8 (0.9) | 1.9 (1.6) | 1.7 (1.4) | 1.3 (1.4) |
| rated | | | | |
| GDS caregiver | 0.6 (1.0) | 3.2 (2.8) | 4.0 (3.3) | 2.0 (2.6) |
| rated | | | | |
| % report a h/o | 7.9 | 29.8 | 34.8 | 19.2 |
| “Depression” | | | | |

GDS, Geriatric Depression Scale;
LMIIA, Wechsler Memory Scale-Revised Logical Memory \StoryADelayed;
MCI-ADL, Alzheimer's Disease Cooperative Study Activities of Daily Living Scale for Mild Cognitive Impairment;
SRTFR, Selective Reminding Task Free Recall Total;
StrI, Stroop Color Task Color-Word Interference Task.

TABLE 8
Model fit

| Model | χ2:df, p | CMIN | RMSEA | CFI |
|---|---|---|---|---|
| 1 | 7.4:9, p = 0.60 | 0.08 | 0.000 | 1.000 |
| 2 | 9.9:9, p = 0.36 | 1.10 | 0.023 | 0.986 |
| 3 | 7.6:8, p = 0.47 | 0.95 | 0.000 | 1.000 |
| 4 | 15.7:15, p = 0.41 | 1.04 | 0.017 | 0.999 |

As was true for d, DEPCOG was a strong independent predictor of CDR-SB (r=−0.91, p<0.001). DEPCOG is labeled moderately strongly by GDSs (r=−0.30, p<0.001) and more strongly by the cognitive measures (range r=0.61, StrI; −0.81, SRTFR; all p<0.001). This model again has excellent fit, and fit marginally less well than Model 1 (Table 8).

d, g′, and DEPCOG were compared as predictors of a wide range of clinical outcomes, bedsides CDR-SB. The latent variable d and DEPCOG were comparably strong predictors of categorical BAP clinical diagnoses, and of our previously reported “δ” dementia endophenotype, constructed from an overlapping but more extensive psychometric battery, and localized to the DMN (Royall et al. (2012) J Alzheimers Dis 30, 639-49) (Table 9). DEPCOG is significantly associated with the subject's history of depressive illness (r=−0.51, p<0.001) as is d (r=−0.30, p<0.001). The latent variable g′ is not, in either model (both p>0.05). DEPCOG is strongly associated with ADL-MCI scores (r=0.69, p<0.001), but slightly less strongly than that measure's loading on d (r=0.77, FIG. 13).

TABLE 9
Latent variable partial correlations with clinical outcomes

| Predictor | Outcome | r* | p |
|---|---|---|---|
| D | CDR-SB | −0.94 | ≦0.001 |
| DEPCOG | CDR-SB | −0.91 | ≦0.001 |
| g′** | CDR-SB | 0.12 | 0.745 |
| d | Diagnosis | −0.81 | ≦0.001 |
| DEPCOG | Diagnosis | −0.99 | ≦0.001 |
| g′ | Diagnosis | 0.22 | 0.627 |
| d | δ† | 0.94 | ≦0.001 |
| DEPCOG | δ | 0.86 | ≦0.001 |
| g′ | δ | −0.15 | 0.736 |
| DEPCOG | ADL-MCI | 0.69 | ≦0.001 |
| g′ | ADL-MCI | −0.24 | 0.655 |
| DEPCOG | Depression | −0.51 | 0.016 |
| d | Depression | −0.30 | 0.001 |
| g′ | Depression | 0.23 | 0.425 |

*partial r, adjusted for covariates: age, gender, and education.;
**g′ from Model 2; adjusted for DEPCOG rather than g′ adjusted for δ score endophenotype from Royall et al. (2012) J Alzheimers Dis 32, 467-78;
†δ from Royall et al. (2012) J Alzheimers Dis 32, 467-78.

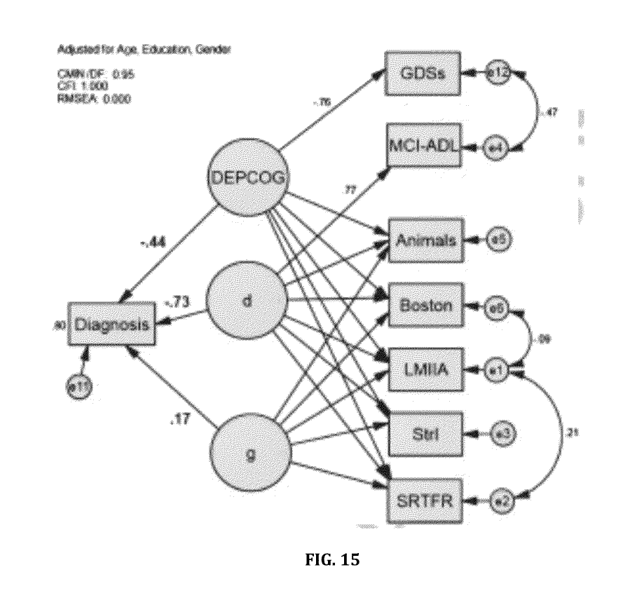

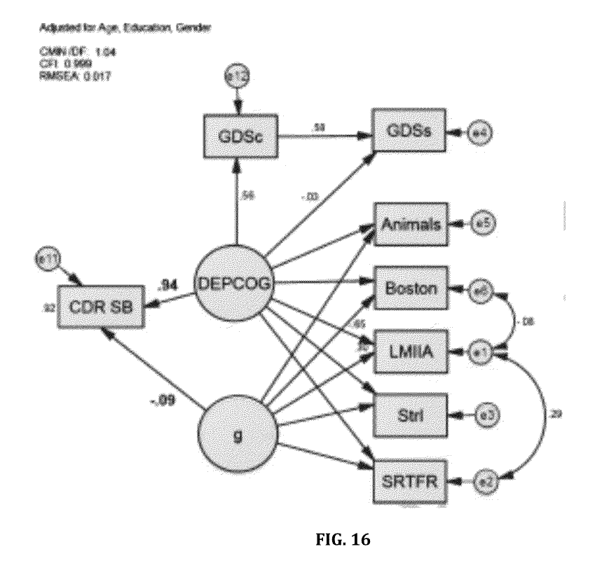

Next the inventors examined d and DEPCOG's independent multivariate associations with BAP clinical diagnoses (FIG. 15). In this model, “d” and “DEPCOG” are orthogonal and are obviously not identical to their unadjusted analogs in Models 1 and 2 (respectively). Adjusting these constructs is desirable in order to demonstrate any residual independent effect of DEPCOG on AD diagnosis. Eighty percent of the variance was explained by DEPCOG, d, g′, and covariates, but only DEPCOG (r=−0.44, p=0.044), d (r=−0.73, p<0.001), and education (r=−0.21, p=0.011) made significant independent contributions. DEPCOG and d attenuate each other relative to their mutually unadjusted associations in Table 9.

Because the DMN is activated by self-referential cognitive tasks, the possibility that DEPCOG's association with dementia status could be mediated through the self-reported nature of depressive symptoms surveys was considered, independent of their symptom content. However, GDSs' loading on DEPCOG is completely mediated by caregiver-rated GDS scores (GDSc), while DEPCOG's association with CDR-SB is unaffected. Therefore, the cognitive correlates of self-reported depression ratings are mediated by their depressive symptom content. This model has excellent fit (Table 8).

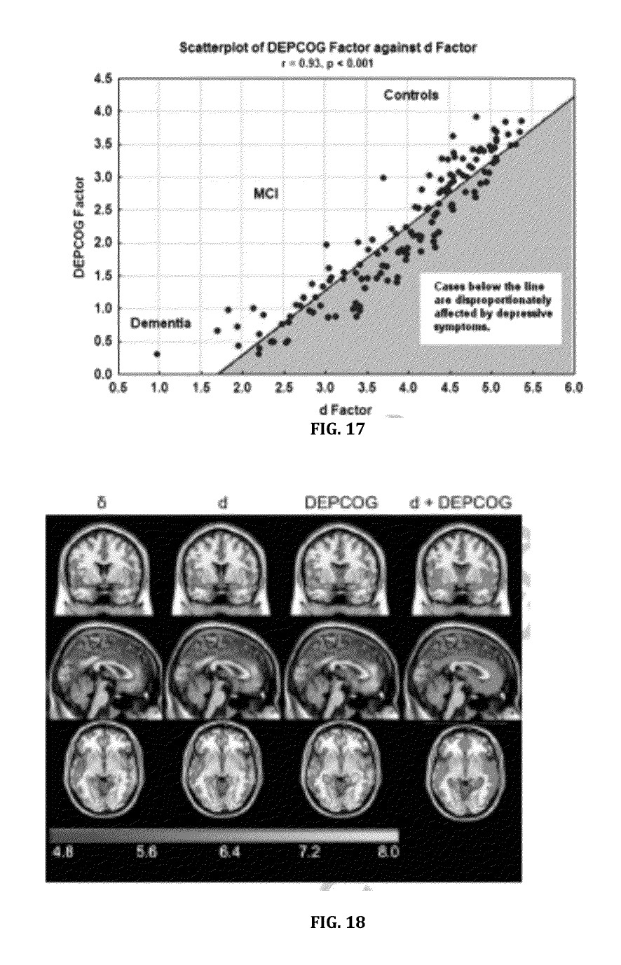

Next, the inventors constructed d and DEPCOG endophenotypes from their age, education, and gender adjusted (but not mutually adjusted) factor loadings. DEPCOG factor scores correlated strongly with d's (r=0.93, p<0.001) (FIG. 17). A significant fraction of the cases are disproportionately affected by the cognitive correlates of depressive symptoms. This presentation appears most common among MCI cases.

FIG. 18 and Table 10 present the locations of peak correlation of gray matter volume with DEPCOG. All associations are adjusted for age, education, and gender (and implicitly g′). Visual inspection of FIG. 18 reveals a strong overlap with elements of the DMN previously associated with d, notably bilateral medial frontal and anterior cingulate gyri, bilateral posterior cingulate and precuneus, and bilateral superior temporal lobe (Table 10).

TABLE 10
Locations of peak correlation of gray matter volume with DEPCOG*

| | | | | | cluster | | |
|---|---|---|---|---|---|---|---|
| Region | BA | x | y | z | size | T | Z |
| Medial frontal and anterior | 10 & 32 | −7 | 45 | 16 | 17101 | 9.4 | >8 |
| cingulate gyri | | | | | | | |
| | | 5 | 47 | −12 | | 8.5 | 7.62 |
| | | 6 | 42 | 16 | | 8.47 | 7.6 |
| Middle and posterior | 31 | 3 | −45 | 30 | 5238 | 8.32 | 7.49 |
| cingulate gyrus | | | | | | | |
| | | 5 | −61 | 21 | | 7.56 | 6.92 |
| | | 8 | −38 | 39 | | 7.53 | 6.89 |
| Transverse and superior | 38 & 13 | 33 | −30 | −11 | 39434 | 10.06 | >8 |
| temporal gyri, & Insula | | | | | | | |
| | | 45 | 1 | −12 | | 9.87 | >8 |
| | | 40 | −5 | 11 | | 9.85 | >8 |
| Superior temporal gyrus | 42 | 62 | −34 | 18 | 283 | 7.07 | 6.53 |
| Middle and superior | 22 | −56 | −8 | −11 | 5313 | 7.71 | 7.03 |
| temporal gyri | | | | | | | |
| | | −57 | −17 | −8 | | 7.65 | 6.98 |
| | | −47 | −12 | −6 | | 7.11 | 6.56 |
| Insula | | −38 | 1 | 6 | 2899 | 8.03 | 7.28 |
| | | −36 | 10 | 4 | | 7.98 | 7.24 |
| | | −38 | −11 | 13 | | 7.58 | 6.93 |
| Hippocampus/parahippocampal | | −23 | −13 | −21 | 4507 | 8.65 | 7.73 |
| gyrus | | | | | | | |
| | | −29 | −35 | −8 | | 7.57 | 6.92 |
| Parahippocampal gyrus | | −21 | 2 | −16 | 575 | 7.57 | 6.92 |
| Middle frontal gyrus | | 23 | 33 | −16 | 462 | 7.41 | 6.8 |
| | | 30 | 52 | 12 | 289 | 7.18 | 6.61 |
| Inferior frontal gyrus | 46 | −44 | 40 | 2 | 221 | 7.00 | 6.47 |
| | | 44 | 41 | 11 | 306 | 6.98 | 6.45 |
| Thalamus | | −7 | 1 | 8 | 389 | 6.84 | 6.34 |
| | | −1 | −2 | 3 | | 6.72 | 6.25 |

*Adjusted for age, gender, education (and implicitly for g′). Higher d scores are associated with greater gray matter volume in these regions.;
†To more precisely define regions of peak association, a higher threshold is used for reporting (FWE 0.001, k > 200).

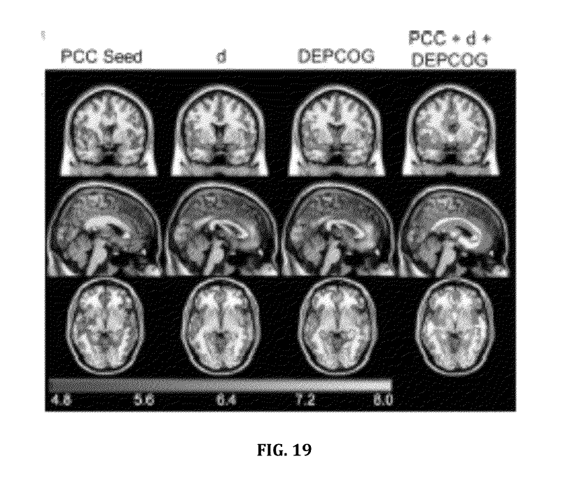

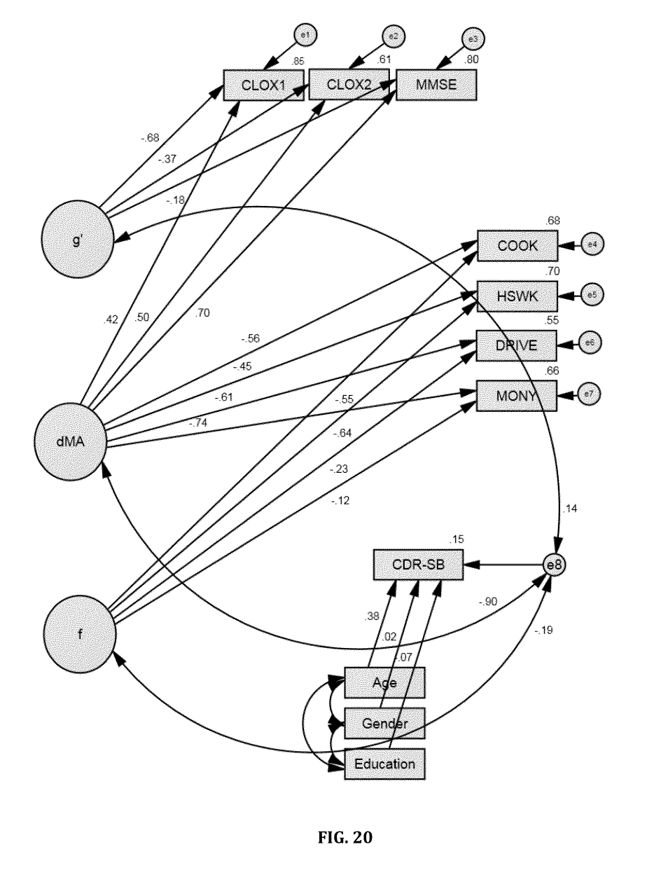

Finally, the inventors examined whether the grey matter atrophy associated with d and DEPCOG is co-localized with posterior cingulate (PCC)-related structures. After placing a seed in that ROI, a network of intercorrelated structures emerged that overlaps with those associated with d and DEPCOG (FIG. 19). All three are co-localized within a subset of the PCC seeded network. Their region of overlap does not include the PCC's thalamic or periventricular corpus callosum connections. Nor does it include d and DEPCOG's hippocampal insular, or precuneus and inferior medio-frontal overlap. Instead, these three networks overlap primarily in DMN-related structures, including again the bilateral anterior cingulate gyri, bilateral posterior cingulate, and bilateral superior temporal lobe.

B. Methods

Sample.

Participants were enrolled in the University of Kansas BAP. Data used in these analyses were from individuals with early-stage AD, defined by a Clinical Dementia Rating (CDR) scale score of 0.5 or 1.0, n=70) or those without dementia (CDR=0, n=76) aged 60 years and older (Buckner et al. (2008) Ann NY Acad Sci 1124, 1-38). Study exclusions have been reported previously (Hughes (1982) Psychiatry 140, 566-72) and briefly include baseline neurologic disease other than AD with the potential to impair cognition, current or past history of diabetes mellitus, recent history of cardiovascular disease, clinically significant depressive symptoms, and magnetic resonance imaging (MRI) exclusions among others. Portions of these data have been reported previously as part of a larger cohort (Burns et al. (2008) Neurol 71, 210-16; Honea et al. (2005) Am J Psychiatry 162, 2233-45; Vidoni et al. (2012) Neurobiol Aging 33, 1624-32). Institutionally approved informed consent was obtained from all participants and their legal representative as appropriate before enrollment into the study.

Clinical Assessment.

The clinical assessment included a semi-structured interview with the participant and a collateral source knowledgeable about the participant. Medications, past medical history, education, demographic information, and family history were collected from the collateral source. Dementia status of the participant was based on clinical evaluation (Morris et al. (2001) Arch Neurol 58, 397-405). Diagnostic criteria for AD require the gradual onset and progression of impairment in memory and at least one other cognitive and functional domain (McKhann et al. (1984) Neurology 34, 939-44). The CDR assesses function in multiple domains and was used to assess dementia severity, such that CDR 0.0 indicates no dementia, CDR 0.5 indicates very mild, and CDR 1.0 indicates mild dementia (Morris (1993) Neurology 43, 2412-14). These methods have a diagnostic accuracy for AD of 93% and have been shown to be accurate in discriminating those with MCI who have early stage AD (Morris (2001) Arch Neurol 58, 397-405; Berg et al. (1998) Arch Neurol 55, 326-35). Problems were encountered when trying to model three diagnostic classes, including the relatively small numbers of AD and MCI cases, and instead modeled “Diagnosis” as AD and MCI (n=70) versus controls (n=76).

Depressive symptoms were assessed using the short Geriatric Depression Scale (GDS) (Sheikh and Yesavage (1986) Clin Gerontologist 5, 165-73; Logsdon and Teri (1995) J Am Geriatr Soc 43, 150-155). Subjects were asked to self-report their depressive symptoms, while their caregivers were asked to assess the subjects' dysphoria. GDS scores range from zero-15. Higher scores are worse. A cut-point of 6-7 best discriminates clinically depressed from non-depressed elderly.

Cognitive Assessment.

A trained psychometrician administered a psychometric test battery that included common measures of memory (Wechsler Memory Scale [WMS]—Revised Logical Memory IA and IIA (Wechsler and Stone (1973) Manual: Wechsler Memory Scale, Psychological Corporation, New York), Free and Cued Selective Reminding Task (Grober et al. (1988) Neurology 38, 900-903), working memory [WMS III Digit Span Forwards and Backwards (Wechsler and Stone (1973) Manual: Wechsler Memory Scale, Psychological Corporation, New York), executive function [Verbal Fluency-Animals (Haenninen et al. (1994) J Am Geriatr Soc 42, 1-4), and Stroop Color-Word Interference Test (Stroop (1935) J Exp Psychol 18, 643-62)). The Mini-Mental State Examination (MMSE) (Folstein et al. (1975) J Psychiatr Res 12, 189-98) was also administered.

Functional Assessment.

The inventors used the Alzheimer's Disease Cooperative Study Activities of Daily Living Scale for Mild Cognitive Impairment (ADCS-ADL) with information collected from the informant. The ADCS-ADL is a well characterized measure of independence in activities of daily living (Galasko et al. (1997) Alzheimer Dis Assoc Disord 11 (Suppl 2), S33-S39). The 18-item measure is heavily weighted toward instrumental activities of daily living (IADL) such as meal preparation, travel outside the home, shopping, and performing household chores. Tasks are scored by increasing level of independence with greater scores reflecting more independence in IADL.

Statistical Approach.

This analysis was performed using AMOS software (Arbuckle (2006) Analysis of Moment Structures-AMOS (Version 7.0) Computer Program, SPSS, Chicago). All observed variables were adjusted for age, gender, and education. Latent variables of interest were constructed from confirmatory factor analyses performed in a structural equation framework. The latent variables d and DEPCOG were uniquely indicated by IADL and GDS scores, respectively. Otherwise, they were derived from an identical cognitive battery that was both more circumscribed and had no overlap with that of previously validated latent variable “δ” (Royall et al. (2012) J Alzheimers Dis 32, 467-78). There was no overlap at all in the indicator variables used to construct DEPCOG and δ. Residual covariances were empirically modeled to optimize model fit. Model parameters were compared across models to ensure that model interpretation remained stable across alternative residual covariance structures.

Missing Data.

Full Information Maximum Likelihood (FIML) methods were used to address missing data. FIML uses the entire observed data matrix to estimate parameters with missing data. In contrast to listwise or pairwise deletion, FIML yields unbiased parameter estimates, preserves the overall power of the analysis, and is arguably superior to alternative methods, e.g., multiple imputation (Schafer and Graham (2002) Psychol Methods 7, 147-177; Graham (2009) Ann Rev Psychol 6, 549-76).

Fit Indices.

The validity of structural models was assessed using three common test statistics. A non-significant chi-square signifies that the data are consistent with the mode (Bollen K A, Long J S (1993) Testing structural equation models. Sage Publications, Thousand Oaks, Calif.). The comparative fit index (CFI), with values ranging between 0 and 1, compares the specified model with a model of no change (Bentler (1990) Psychol Bull 107, 238-46). CFI values below 0.95 suggest model misspecification. Values of 0.95 or greater indicate adequate to excellent fit. A root mean square error of approximation (RMSEA) of 0.05 or less indicates a close fit to the data, with models below 0.05 considered “good” fit, and up to 0.08 as “acceptable” (Browne and Cudeck (1993) Alternative ways of assessing model fit. In Testing Structural Equation Models, Bollen Long J S, eds. Sage Publications, Thousand Oaks, Calif. pp. 136-62). All three fit statistics should be simultaneously considered to assess the adequacy of the models to the data.

Neuroimaging.

Baseline and follow-up whole brain structural MRI data were obtained using a Siemens 3.0 Tesla Allegra MRI Scanner. High-resolution T1 weighted anatomical images were acquired (magnetization-prepared rapid gradient echo [MPRAGE]; 1×1×1 mm³ voxels, repetition time [TR]=2500, echo time [TE]=4.38 ms, inversion time [TI]=1,100 ms, field of view=256×256, flip angle=8°). Data analysis was performed using the VBM5toolbox (URL dbm.neuro.uni-jena.de), an extension of the SPM5 algorithms (Wellcome Department of Cognitive Neurology, London, UK) running under MATLAB 7.1 (The Math-Works, Natick, Mass., USA).

Voxel-based morphometry (VBM) is a method for detecting differences in the volume of brain matter. Structural image processing method for VBM is detailed elsewhere (Burns et al. (2008) Neurol 71, 210-16; Honea et al. (2009) Alzheimer Dis Assoc Disord 23, 188-97). Briefly, tissue classification, image registration, and MRI inhomogeneity bias correction were performed as part of the unified segmentation approach implemented in SPM5 (Ashburner and Friston (2005) Neuroimage 26, 839-51). The inventors used the Hidden Markov Field (HMRF) model on the estimated tissue maps (3×3×3 mm³). Estimated tissue probability maps were written without making use of the International Consortium for Brain Mapping tissue priors to avoid a segmentation bias (Gaser et al. (2007) Neuroimage 36 Suppl 1, S68). Images were then modulated and saved using affine registration plus non-linear spatial normalization (Wilke et al. (2008) Neuroimage 41, 903-13). The resulting gray matter volume maps were smoothed with a 10 mm FWHM Gaussian kernel before statistical analysis.

Imaging Statistics.

The inventors used a multiple regression model in SPM5 with age, education, and gender as covariates (age and education centered on the mean). DEPCOG scores are also implicitly adjusted for g′ factor scores. The absolute threshold masking was set at 0.10 to restrict each analysis to gray matter. Of primary interest was the relationship of DEPCOG factor scores with regional gray matter volume, independent of the remaining regressors. Results were considered significant at p<0.05 [family-wise error corrected (FWE)], with clusters exceeding 50 voxels. Peak voxels are reported with reference to the MNI standard space and anatomic labels are reported with reference to the computerized Talairach Daemon (Lancaster et al. (1997) Hum Brain Mapp 5, 238-42) within the Pickatlas (Maldjian et al. (2003) Neuroimage 19, 1233-39). DEPCOG's regional gray matter volume correlates are compared in FIGS. 18 and 19 to those of d and the previously validated original model “δ” (Royall et al. (2012) J Alzheimers Dis 32, 467-78).

Oh et al. (2011, Neuroimage 54, 187-95) have demonstrated that PiB burden in non-demented older persons is associated with atrophy in the posterior cingulate. When a seed was placed in that structure, an inter-related set of structures emerged. We tested whether the grey matter atrophy associated with our latent constructs is co-localized with the same structures. A multiple regression model was used in SPM5 with age, education, and gender as covariates (age and education centered on the mean). d and DEPCOG scores are also implicitly adjusted for g′ factor scores. The absolute threshold masking was set at 0.10 to restrict each analysis to gray matter. The primary interest was the relationship of DEPCOG factor scores with regional gray matter volume, independent of the remaining regressors. Relative brain volume was extracted from a bilateral posterior cingulate ROI (4 mm spheres at −10, −38, 30 and 10, −38, 30) (Oh et al. (2011) Neuroimage 54, 187-95) and regressed the relative gray matter volume corrected for age, gender, and education as has been done recently by Monembault et al. (2012 Neuroimage 63, 754-759).

### Example 4

### Validation of a Latent Construct for Dementia Case-Finding in Mexican-Americans

The inventors have constructed a latent dementia proxy, “δ”, and validated it in several datasets, including well characterized subjects participating in the Texas Alzheimer's Research and Care Consortium (TARCC) study.

The latent variable δ represents the “cognitive correlates of functional status”. It is uniquely related to dementia severity as measured by the Clinical Dementia Rating scale Sum of Boxes (CDR-SB) and accurately distinguishes cases with Alzheimer's disease (AD), and Mild Cognitive Impairment (MCI) from each other, and from controls.

The latent variable δ can be constructed from almost any ad hoc combination of cognitive and functional status measures. It is also relatively immune to measurement error, including cultural, linguistic or educational biases. These properties make latent variables an attractive solution for dementia case-finding in rural or minority populations. In this example the inventors studied the assessment needed to construct 6, and validate the resulting latent variable (dMA) in Mexican-American (MA) TARCC subjects.

A. Results

Descriptive statistics are presented in Tables 11-13. The TARCC sample is relatively highly educated, and has a slight preponderance of females. 26.6% of subjects reported Hispanic ethnicity. The AD group was significantly less well educated, and more impaired on multiple measures relative to the MCI cases, but no older. The MCI group was more impaired on multiple measures relative to MCI cases, excepting CLOX2. AD cases were significantly more impaired on IADL's than MCI cases, who were indistinguishable from controls.

TABLE 11
Descriptive Statistics Total Sample

| | | | | Post hoc tests | | | | | | |
|---|---|---|---|---|---|---|---|---|---|---|
| | | Total | | AD | | MCI | | Controls | | Main |
| | Variable | N = 2017 | | N = 920 | | N = 277 | | N = 819 | | Effect |
| | N | Mean (SD) | | Mean (SD) | | Mean (SD) | | Mean (SD) | | P |
| Gender (% female) | 2016 | 60% | | 57%^{#} | | 55%^{#} | | 65%^{‡†} | | ≦0.001 |
| Ethnicity (% Hispanic) | 2016 | 26.6% | | 9.0%^{#‡} | | 37.6%^{†} | | 42.6%^{†} | | ≦0.001 |
| Age at Visit | 2016 | 72.59 | (9.4) | 76.11 | (8.3)^{#} | 73.38 | (9.0) | 68.38 | (9.1)^{†} | ≦0.001 |
| Education | 2016 | 13.87 | (3.8) | 14.16 | (3.6)^{#‡} | 13.45 | (3.5)^{#†} | 13.68 | (4.1)^{‡†} | 0.004 |
| CLOX1 | 931 | 11.57 | (2.7) | 9.57 | (3.3)^{#‡} | 11.63 | (2.5)^{#†} | 12.71 | (1.6)^{‡†} | ≦0.001 |
| CLOX2 | 926 | 13.28 | (1.8) | 12.08 | (2.4)^{#‡} | 13.53 | (1.3)^{†} | 13.84 | (1.2)^{†} | ≦0.001 |
| MMSE | 2016 | 25.06 | (5.3) | 20.99 | (5.2)^{#‡} | 27.20 | (2.4)^{#†} | 28.90 | (1.7)^{‡†} | ≦0.001 |
| CDR (Sum of Boxes) | 2011 | 2.93 | (3.8) | 6.08 | (3.5)^{#‡} | 1.12 | (0.8)^{#†} | 0.02 | (0.1)^{‡†} | ≦0.001 |
| GDS (30 item) | 1719 | 5.04 | (4.8) | 5.61 | (4.8)^{#‡} | 6.69 | (5.8)^{#†} | 3.90 | (4.1)^{‡†} | ≦0.001 |
| IADL (Summed) | 1501 | 10.36 | (5.0) | 15.00 | (6.0)^{#‡} | 8.37 | (2.3)^{†} | 7.82 | (0.9)^{†} | ≦0.001 |

| | | | | |
|---|---|---|---|---|
| Complete Cases | 911 | 243 | 242 | 425 |

CDR = Clinical Dementia Rating scale; GDS = Geriatric Depression Scale; IADL = Instrumental Activities of Daily Living; MMSE = Mini-mental State Exam; SD = standard deviation.
^{†}p < 0.05 vs. AD by Tukey's HSD for unequal n's.
^{‡}p < 0.05 vs. MCI by Tukey's HSD for unequal n's.
^{#}p < 0.05 vs. Controls by Tukey's HSD for unequal n's.

TABLE 12
Descriptive Statistics MA Subjects

| | | | | Post hoc tests | | | | | | |
|---|---|---|---|---|---|---|---|---|---|---|
| | | Total | | AD | | MCI | | Controls | | Main |
| | Variable | N = 537 | | N = 83 | | N = 104 | | N = 349 | | Effect |
| | N | Mean (SD) | | Mean (SD) | | Mean (SD) | | Mean (SD) | | P |
| Gender (% female) | 537 | 62% | | 60% | | 54% | | 64% | | 0.159 |
| Age at Visit | 537 | 67.94 | (9.2) | 75.63 | (7.6)^{#‡} | 72.27 | (9.0)^{#†} | 64.85 | (7.9)^{‡†} | ≦0.001 |
| Education | 537 | 11.16 | (4.6) | 9.73 | (5.2)^{‡} | 11.55 | (4.1)^{†} | 11.38 | (4.6) | 0.009 |
| CLOX1 | 466 | 11.66 | (2.7) | 7.93 | (3.4)^{#‡} | 10.69 | (2.9)^{#†} | 12.59 | (1.6)^{‡†} | ≦0.001 |
| CLOX2 | 466 | 13.31 | (1.7) | 10.98 | (2.8)^{#‡} | 13.18 | (1.4)^{#†} | 13.75 | (1.2)^{‡†} | ≦0.001 |
| MMSE | 537 | 26.63 | (4.4) | 19.07 | (5.1)^{#‡} | 26.94 | (2.4)^{#†} | 28.34 | (2.2)^{‡†} | ≦0.001 |
| CDR (Sum of Boxes) | 537 | 1.09 | (2.5) | 5.80 | (3.4)^{#‡} | 0.94 | (0.60^{#†} | 0.01 | (0.2)^{‡†} | ≦0.001 |
| GDS (30 item) | 524 | 5.84 | (5.5) | 8.52 | (5.5)^{#} | 7.99 | (6.6)^{#} | 4.59 | (4.6)^{‡†} | ≦0.001 |
| IADL (Summed) | 522 | 9.07 | (3.8) | 15.99 | (6.4)^{#‡} | 8.22 | (2.2)^{†} | 7.89 | (0.9)^{†} | ≦0.001 |

| | | | | |
|---|---|---|---|---|
| Complete Cases | 463 | 52 | 94 | 316 |

CDR = Clinical Dementia Rating scale; GDS = Geriatric Depression Scale; IADL = Instrumental Activities of Daily Living; MMSE = Mini-mental State Exam; SD = standard deviation.
^{†}p < 0.05 vs. AD by Tukey's HSD for unequal n's.
^{‡}p < 0.05 vs. MCI by Tukey's HSD for unequal n's.
^{#}p < 0.05 vs. Controls by Tukey's HSD for unequal n's.

TABLE 13
Descriptive Statistics NHW Subjects

| | | | | Post hoc tests | | | | | | |
|---|---|---|---|---|---|---|---|---|---|---|
| | | Total | | AD | | MCI | | Controls | | Main |
| | Variable | N = 1479 | | N = 836 | | N = 173 | | N = 470 | | Effect |
| | N | Mean (SD) | | Mean (SD) | | Mean (SD) | | Mean (SD) | | P |
| Gender (% female) | 1479 | 59% | | 50%^{#} | | 50% | | 48%^{†} | | 0.004 |
| Age at Visit | 1479 | 74.28 | (8.9) | 76.17 | (8.3)^{#} | 74.05 | (8.9)^{#} | 71.00 | (9.0)^{†‡} | ≦0.001 |
| Education | 1479 | 14.85 | (2.9) | 14.60 | (3.0)^{#} | 14.60 | (2.5)^{#} | 15.38 | (2.6)^{†‡} | ≦0.001 |
| CLOX1 | 464 | 11.47 | (1.8) | 9.99 | (3.1)^{#‡} | 12.20 | (1.9)^{#†} | 13.05 | (1.3)^{†‡} | ≦0.001 |
| CLOX2 | 459 | 13.25 | (2.7) | 12.37 | (2.1)^{#‡} | 13.75 | (1.2)^{†} | 14.08 | (1.0)^{†} | ≦0.001 |
| MMSE | 1473 | 24.49 | (5.5) | 21.18 | (5.1)^{#‡} | 27.36 | (2.4)^{#†} | 29.32 | (0.9)^{†‡} | ≦0.001 |
| CDR (Sum of Boxes) | 1479 | 3.60 | (3.9) | 6.11 | (3.5)^{#‡} | 1.23 | (0.9)^{#†} | 0.02 | (0.1)^{†‡} | ≦0.001 |
| GDS (30 item) | 1194 | 4.68 | (4.4) | 5.24 | (4.6)^{#} | 5.89 | (5.1)^{#} | 3.32 | (3.4)^{†‡} | ≦0.001 |
| IADL (Summed) | 978 | 11.04 | (5.4) | 14.84 | (5.9)^{#‡} | 8.46 | (2.3)^{†} | 7.75 | (1.0)^{†} | ≦0.001 |

| | | | | |
|---|---|---|---|---|
| Complete Cases | 448 | 191 | 148 | 109 |

CDR = Clinical Dementia Rating scale; GDS = Geriatric Depression Scale; IADL = Instrumental Activities of Daily Living; MMSE = Mini-mental State Exam; SD = standard deviation.
^{†}p < 0.05 vs. AD by Tukey's HSD for unequal n's.
^{‡}p < 0.05 vs. MCI by Tukey's HSD for unequal n's.
^{#}p < 0.05 vs. Controls by Tukey's HSD for unequal n's.

The model used to construct fMA, gMA and dMA had excellent fit (χ²:df; 36.87:14, p≦0.001; CFI=0.995; RMSEA=0.028). In MA subjects, the cognitive measures loaded significantly and inversely on gMA, ranging from (r=−0.18 to −0.68, all p<0.001). The IADL items loaded significantly and inversely on fMA, ranging from (r=−0.12 to −0.64, all p<0.02). Both cognitive measures and IADL items loaded significantly on dMA, ranging from (r=0.42 to 0.74, all p<0.001). The cognitive indicators were positively associated with dMA scores. The IADL items, which are inversely scaled, were inversely related to dMA scores. Thus, higher dMA scores indicate better cognitive performance and functional status, and a lower risk of clinical dementia.

dMA's factor loadings exhibited factor equivalence when stratified across two random subsets (χ²:df=149.3:21 vs. 36.87:14 when unconstrained, p≦0.05 by chi sq tables), but not when stratified by ethnicity (χ²:df=149.4:21 vs. 36.87:14, p≦0.05 by chi sq tables).

In multivariate regression models adjusted for age, gender, and education, the latent variable dMA was strongly associated with CDR-SB (r=−0.89, p≦0.001), as well as with the previously validated d homologs: δ (r=0.85, p≦0.001), and dCDR (r=0.81, p≦0.001). All of these associations were slightly, but significantly stronger in non-Hispanics than in MA (Table 14).

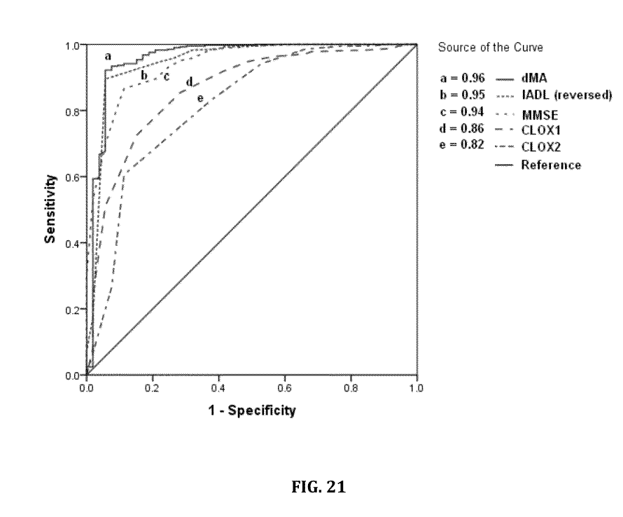

FIG. 21 presents an ROC analysis of dMA and each of its raw indicator variables as predictors of dementia in MA participants. dMA incrementally improves upon the discriminatory power of its indicators.

Table 15 presents an ROC analysis of dMA scores as predictors of adjudicated TARCC clinical diagnoses in MA subjects. Its AUC for the discrimination of AD v. controls was 0.964 in MA. Its AUC for this discrimination in non-Hispanics was slightly stronger (Table 15). Its AUC for the discrimination of AD v. MCI was 0.938 in MA. Its AUC for this discrimination in non-Hispanics was slightly weaker.

TABLE 14
Partial Correlations (r) Between dMA Scores and Descriptors of
Dementia Severity by Ethnicity

| | MA | NHW |
|---|---|---|
| CDR-SB | −0.89 | −0.91 |
| δ¹ | 0.81 | 0.84 |
| dCDR² | 0.85 | 0.90 |

¹Royall, Palmer & O'Bryant, 2012
²Royall & Palmer, in review

TABLE 15
ROC Analysis of dMA as a Predictor of
Adjudicated Clinical Dementia Status, Stratified by Ethnicity

| | MA | NHW |
|---|---|---|
| Discrimination | AUC | AUC |
| AD v. Controls | 0.964 | 0.974 |
| AD v. MCI | 0.938 | 0.904 |
| MCI v. Controls | 0.693 | 0.671 |
| AD v. All | 0.958 | 0.934 |

AD = Alzheimer's Disease;
AUC = Area Under the Curve;
MCI = Mild Cognitive Impairment;
NHW = nonHispanic Whites;
ROC = Receiver Operating Curve.

The latent variable dMA's AUC for the discrimination of MCI v. controls was only 0.693. This probably reflects measurement ceiling and/or floor effects among dMS's indicator variables among early MCI cases and controls, and appears to limit dMA's utility for pre-dementia screening. Therefore, the inventors only examined the dMA threshold that best distinguished AD cases from all others. In MA, this appeared to be at dMA=2.0605. This threshold achieved a sensitivity of 0.94 for the detection of MA AD cases and a specificity of 0.95. It correctly classified 90.3% of MA AD cases, and 99% of controls (χ²: 348 (1) F=171.91; p≦0.001).

Finally, the inventors examined dMA's discrimination of AD from all other diagnoses in MA by discriminant analysis. dMA correctly classified 90.3% of AD cases and 92.0% of non-AD cases and controls (91.9% overall). The model was significant [Wilks' Lambda=0.629: F (1,440)=259.81, p≦0.001]. The latent variable dMA discriminated less well among NHW [74.9% of AD cases correctly classified vs. 93.9% of non-AD cases and controls (86.5% overall)].

B. Methods

Subjects:

The subjects represent visit 1 data from the Texas Alzheimer's Research Consoritum (TARCC) cohort (circa 2008-2011). Subjects included N=2016 TARCC participants (920 cases of AD, 277 MCI cases, and 819 controls). The methodology of the TARCC project has been described in detail elsewhere (Waring et al. (2008) Texas Pub Health J 60, 9-13). Each participant underwent a standardized annual examination at their respective evaluation site that includes a medical evaluation, neuropsychological testing, and clinical interview. Diagnosis of AD status was based on National Institute for Neurological Communicative Disorders and Stroke—Alzheimer's Disease and Related Disorders Association (NINCDS—ADRDA) criteria (McKhann et al. (1984) Neurology 34, 939-944). Controls performed within normal limits on their psychometric assessments. Institutional Review Board approval was obtained at each site and written informed consent was obtained for all participants.

Clinical Variables:

Depressive symptoms were assessed using the 30-item Geriatric Depression Scale (GDS) (Sheikh and Yesavage (1986) Clin Gerontologist 5, 165-173; Maixner et al. (1995) Am J Geriatr Psychiatry 3, 60-67). GDS scores range from zero-15. Higher scores are worse. A cut-point of 9-10 best discriminates clinically depressed from non-depressed elderly.

Instrumental Activities of Daily Living (IADL) were assessed using care-giver ratings (Lawton and Brody (1969) Gerontologist 9, 179-86). The ability to use the telephone (TEL), shopping (SHOP), food preparation (COOK), housekeeping (HSWK), laundry (WASH) use of transportation (DRIVE) ability to handle finances (MONY) and responsibility for medication adherence (MEDS) were each rated on a Likert scale ranging from 0 (no impairment) to 3 (specific incapacity).

The Clinical Dementia Rating Scale sum of boxes (CDR-SB) (Hughes et al. (1982) Br J Psychiatry 140, 566-72): The CDR was used to evaluate dementia severity. This rating assesses the patient's cognitive ability to function in six domains—memory, orientation, judgment and problem solving, community affairs, home and hobbies and personal care. Information is collected during an interview with the patient's caregiver. Optimal CDR-SB ranges corresponding to global CDR scores are 0.5-4.0, for a global score of 0.5, 4.5-9.0, for a global score of 1.0, 9.5-15.5, for a global score of 2.0, and 16.0-18.0, for a global score of 3.0 (O'Bryant et al. (2008) Arch Neurol 65, 1091-95).

The inventors also used two previously validated latent variable proxies for dementia severity. The latent variable δ was constructed from an extensive set of psychometric measures as previously described (Royall et al. (2012) Journal of Alzheimer's Disease 30, 639-49). δ has an AUC of 0.942 for the discrimination between AD cases and controls in TARCC. dCDR is composed of a reduced set of formal psychometric measures, but uses CDR-SB instead of caregiver rated IADL, as its target indicator. It achieves superior discriminations relative to δ (i.e., AD v. Controls AUC=0.989; AD v. MCI=0.938; Controls v. all others=0.926; MCI v. Controls=0.830.

Cognitive Battery:

The MMSE (Folstein et al. (1975) J Psychiatry Res 12, 189-98) is a well known and widely used test for screening cognitive impairment (Royall et al. (2003) International Journal of Geriatric Psychiatry 18:135-41). Scores range from 0 to 30. Scores less than 24 reflect cognitive impairment. The MMSE has significant educational and cultural biases.

CLOX (Royall et al. Journal of Neurology, Neurosurgery and Psychiatry 64:588-594): The CLOX is a brief ECF measure based on a clock-drawing task and is divided into two parts. CLOX1 is an unprompted task that is sensitive to executive control. CLOX2 is a copied version that is less dependent on executive skills. CLOX1 is more ‘executive’ than other comparable CDT's (Royall et al. Journals of Gerontology: Psychological Sciences 54B:P328-33). Each CLOX subtest is scored on a 15-point scale. Lower CLOX scores are impaired. Cut-points of 10/15 (CLOX1) and 12/15 (CLOX2) represent the 5th percentiles for young adult controls.

CLOX has been validated in MA populations (Royall et al. (2003) International Journal of Geriatric Psychiatry 18:135-41). Socio-demographic variables, including acculturation and language of CLOX performance, explain only 8% of CLOX1 variance, and 6% of CLOX2 variance. Language of CLOX presentation, income and gender had no significant independent effects on either CLOX subtest.

The inventors have examined CLOX performance in a large population-based sample of n=1165 community dwelling MA adults in five southwestern states (mean age=71.4±5.3 years) as part of the Hispanic Established Population for Epidemiological Studies in the Elderly (HEPESE) (Royall et al. (2004) International Journal of Geriatric Psychiatry, 19:926-34). CLOX1 is far more sensitive to cognitive impairment than are either the MMSE or CLOX2. 59.3% failed CLOX1 at 10/15. 27.7% failed the MMSE at/30. 31.1% failed CLOX2 at 12/15.

Statistical Analyses:

The latent variables of interest were constructed from confirmatory factor analyses performed in a structural equation modeling (SEM) framework using Analysis of Moment Structures (AMOS) software (Arbuckle J L (2006) Analysis of Moment Structures-AMOS (Version 7.0) [Computer Program], SPSS, Chicago). The model was stratified by ethnicity, and its factor weights examined separately within MA and NHW groups. Three latent variables representing the cognitive correlates of functional status (i.e., “δ”), g′ (i.e., δ's residual in Spearman's g) and “f” (i.e., the shared variance in IADL not associated with cognition) were defined. The orthogonality of these latent constructs was confirmed empirically. Residual covariances were estimated explicitly for each observed measure, and assumed to be uncorrelated amongst the latent variables' indicators. All observed measures, latent indicators and outcomes, were adjusted for age, gender and education.

The latent variables of interest were compared for their individual correlations with demographic adjusted CDR-SB within ethnic subgroups. The latent variable “dMA” was defined by δ's factor loadings in MA subjects. Its factor equivalence, across ethnicity and randomly selected subsets of TARCC's sample, was tested by constraining its indicator loadings to be equal across groups and then comparing the fit to an unconstrained model. dMA was then extracted as a dummy variable, and correlated with CDR-SB, δ and dCDR in demographic adjusted multivariate regression models. Finally, dMA was validated by Receiver Operating Characteristic (ROC) analyses. In these analyses, stratified by ethnicity, dMA was used without covariates to predict TARCC adjudicated dementia status. An optimal dMA threshold for the discrimination of AD v. all others was selected and tested by χ².

Missing Data:

Some variables (i.e., CLOX and IADL) have not been used consistently since TARCC's inception, and have considerably smaller sample sizes. However, only the ROC were limited to complete cases. Elsewhere, the inventors used Full Information Maximum Likelihood (FIML) methods to address missing data. FIML uses the entire observed data matrix to estimate parameters with missing data. In contrast to listwise or pairwise deletion, FIML yields unbiased parameter estimates, preserves the overall power of the analysis, and is arguably superior to alternative methods, e.g., multiple imputation (Schafer and Graham (2002) Psychol Methods, 7:147-77; Graham (2009) Ann Rev Psychol 6: 549-76).

Fit Indices:

The validity of structural models was assessed using two common test statistics. A non-significant chi-square signifies that the data are consistent with the model (Bollen and Long (1993) Testing Structural Equation Model. Sage Publications, Thousand Oaks, Calif.). The comparative fit index (CFI), with values ranging between 0 and 1, compares the specified model with a model of no change (Bentler (1990) Psychol Bull 107, 238-46). CFI values below 0.95 suggest model misspecification. Values of 0.95 or greater indicate adequate to excellent fit. A root mean square error of approximation (RMSEA) of 0.05 or less indicates a close fit to the data, with models below 0.05 considered “good” fit, and up to 0.08 as “acceptable” (Browne and Cudeck (1993) Alternative ways of assessing model fit. In Testing structural equation models, Bollen K A, Long J S, eds. Sage Publications, Thousand Oaks, Calif., pp. 136-162). All three fit statistics should be simultaneously considered to assess the adequacy of the models to the data.

ROC Curves:

The diagnostic performance or accuracy of a test to discriminate diseased from normal cases can be evaluated using ROC curve analysis (Metz (1978) Sem Nuc Med 8: 283-98; Zweig and Campbell (1993) Clin Chem 39, 561-77). Briefly the true positive rate (Sensitivity) is plotted as a function of the false positive rate (1.00-Specificity) for different cut-off points of a parameter. Each point on the ROC curve represents a sensitivity/specificity pairing corresponding to a particular decision threshold. The area under the ROC curve (AUC) is a measure of how well a parameter can distinguish between two diagnostic groups (diseased/normal).

Cross-group differences were tested using post-hoc tests (i.e., Tukey's Honest Significant Difference for unequal n's) (HSD). The analysis was performed in Statistical Package for the Social Sciences (SPSS) (PASW Statistics 18, Release Version 18.0.0, SPSS, Inc., 2009, Chicago, Ill.).

1. A method of evaluating a data set having a first and second type of assessment by including in a structural equation model a hybrid variable that is related to the covariance between the variance of the first and second type of assessment, wherein the hybrid variable is used to determine a score that is compared to a known scale to classify an outcome.

2. The method of claim 1, wherein the assessment is of dementia status of a subject comprising a hybrid variable defined as a cognitive-functional correlate score (“d score”) that is indicative of covariance between a first cognitive and a second functional status performance assessments of a subject.

3. The method of claim 2, wherein the known scale is an optimal d score for diagnosis of Alzheimer's disease, mild cognitive impairment (MCI), and normal cognition from a validation cohort.

4. A method for evaluating the effectiveness of a therapeutic comprising:
(a) determining a first cognitive-functional correlate score indicative of covariance between cognitive performance assessment and functional performance assessments of a subject;
(b) administering a therapeutic to a subject;
(c) determining a second cognitive-functional correlate score indicative of covariance between cognitive performance assessment and functional performance assessments of a subject; and
(d) comparing the first and second cognitive-functional correlate scores, wherein a relative change in the first and second cognitive-functional correlate score is indicative of the effectiveness of the therapeutic.

5. A method, comprising:
constructing, by a computing device, a score based on a hybrid latent variable generated by a structural equation model that is related to the covariance two or more variances of two or more assessment measures; and classifying one or more outcome based on comparing the constructed score to a known scale constructed from scores of a validation cohort.

6. The method of claim 5, wherein the hybrid latent variable score is a cognitive-functional latent variable score (d score).

7. A method for assessing a condition in an individual relative to a validated cohort comprising:
(a) selecting (i) a battery of behavioral measures of a subject, and (ii) one or more measures of a target condition or disease;
(b) constructing (i) a first latent factor related to variance of the behavioral measures (ii) a second latent factor related to variance of the target measures, and (iii) a third hybrid factor related to covariance of the behavioral measures and the target measures using structural equation modeling (SEM);
(c) determining the hybrid factor loadings on a validation cohort and using the loading to export a score for each individual in a validation cohort;
(d) selecting score thresholds based on the validation cohort;
(e) applying the score threshold to a score obtained from the individual being assessed, wherein the score for the individual is obtained by administering the same set of measures used to construct the hybrid factor in the validation cohort where the individual's score is compared to the score thresholds of the validation cohort.

8. The method of claim 7, wherein the behavioral measure comprise verbal measures.

9. The method of claim 7, wherein a battery of non-proprietary measures are selected.

10. The method of claim 7, wherein a battery of bedside measures are selected.

11. The method of claim 7, wherein the target condition or disease is a diagnosis, mood state, behavior, or biomarker related to the selected behavioral measures.

12. The method of claim 7, wherein optimal score thresholds are selected by Receiver Operating Curve (ROC) analysis of determinations of the same population used to construct the hybrid latent factor.

13. The method of claim 7, wherein operations for the method are at least in part executed on a phone, tablet, computer, or internet-based server.

14. A system, comprising:
(a) at least one processor; and
(b) a memory coupled to the at least one processor, the memory configured to store program instructions executable by the at least one processor to cause the system to:
(i) construct a structural equation model having a hybrid latent variable related to a covariance between two or more variances related to two or more assessments or measurements; and (ii) classify one or more outcome based on a score derived from the hybrid latent variable.

15. A tangible computer-readable storage medium having program instructions stored thereon that, upon execution by one or more computer systems, cause the one or more computer systems to:
(a) construct a structural equation model having a hybrid latent variable related to a covariance between two or more variances related to two or more assessments or measurements; and
(b) classify one or more outcome based on a score derived from the hybrid latent variable.
